SEQUENCE LISTING (32 sequences)

SEQ ID NO: 1 - protein, 17 aa, Artificial Sequence
  Signal peptide of ACE2
MSSSSWLLLSLVAVTAA

SEQ ID NO: 2 - protein, 598 aa, Artificial Sequence
  ACE2 protein sequence aa 18-615
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 185 aa, Artificial Sequence
  CD3epsilon
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - protein, 20 aa, Artificial Sequence
  CR3022 light chain signal peptide
MVLQTQVFISLLLWISGAYG

SEQ ID NO: 5 - protein, 113 aa, Artificial Sequence
  VL CR3022 scFv aa 18 to 133
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 6 - protein, 117 aa, Artificial Sequence
  VH CR3022 scFv aa 20 to 136
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTV

SEQ ID NO: 7 - protein, 5 aa, Artificial Sequence
  G4S linker peptide
GGGGS

SEQ ID NO: 8 - protein, 18 aa, Artificial Sequence
  T2A peptide
EGRGSLLTCGDVEENPGP

SEQ ID NO: 9 - protein, 252 aa, Artificial Sequence
  GFP
MESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 10 - protein, 4 aa, Artificial Sequence
  peptide linker
GGSG

SEQ ID NO: 11 - protein, 18 aa, Artificial Sequence
  peptide linker
GSTSGSGKPGSGEGSTKG

SEQ ID NO: 12 - protein, 8 aa, Artificial Sequence
  peptide linker
LDGSGGDV

SEQ ID NO: 13 - protein, 19 aa, Artificial Sequence
  CR3022 heavy chain signal peptide
MGSTAILALLLAVLQGVCA

SEQ ID NO: 14 - protein, 19 aa, Artificial Sequence
  Ig-alpha chain signal peptide
MGWSCIMLFLAATATGVHS

SEQ ID NO: 15 - protein, 21 aa, Artificial Sequence
  CD8alpha signal peptide
MALPVTALLLPLALLLHAARP

SEQ ID NO: 16 - protein, 17 aa, Artificial Sequence
  GM-CSF signal peptide
MWLQSLLLLGTVACSIS

SEQ ID NO: 17 - protein, 20 aa, Artificial Sequence
  TCRalpha signal peptide T1
MNTSPALVTVMLLFMLERTH

SEQ ID NO: 18 - protein, 28 aa, Artificial Sequence
  TCRalpha signal peptide OT1
MDKILTATFLLLGLHLAGVNGQQQEKRD

SEQ ID NO: 19 - protein, 20 aa, Artificial Sequence
  TCRbeta signal peptide T1
MGSRLFFVLSSLLCSKHMEA

SEQ ID NO: 20 - protein, 29 aa, Artificial Sequence
  TCRbeta signal peptide OT1
MSNTVLADSAWGITLLSWVTVFLLGTSSA

SEQ ID NO: 21 - protein, 39 aa, Artificial Sequence
  TCRgamma signal peptide
MKNPGSQALLPLYLPWEANLADENPLLKVVIFLCLLTFG

SEQ ID NO: 22 - protein, 20 aa, Artificial Sequence
  TCRdelta signal peptide
MQRNLGAVLGILWVQICWVR

SEQ ID NO: 23 - protein, 24 aa, Artificial Sequence
  HLA-A signal peptide
MAVMAPRTLLLLLSGALALTQTWA

SEQ ID NO: 24 - protein, 19 aa, Artificial Sequence
  linker peptide
PNGASQSSSASHTGSAPGS

SEQ ID NO: 25 - protein, 18 aa, Artificial Sequence
  linker peptide
RPLSYRPPFPFGFPSVRP

SEQ ID NO: 26 - DNA, 50 bp, Artificial Sequence
  Primer
atccaagctgtgaccggcgcctactctagagccaccatgtcaagctcttc

SEQ ID NO: 27 - DNA, 70 bp, Artificial Sequence
  Primer
gatgccgcccatttcctcgttgccatcctcgagggatcctccgccgccgtctgcatatggactccagtcg

SEQ ID NO: 28 - DNA, 100 bp, Artificial Sequence
  Primer
gatgccgcccatttcctcgttgccatcctcgagggatcctccgccgccacttcctccgccaccagaaccaccgcctccgtctgcatatggactccagtcg

SEQ ID NO: 29 - DNA, 63 bp, Artificial Sequence
  Primer
tacagatccaagctgtgaccggcgcctactctagagccgccaccatggtgttgcagacccagg

SEQ ID NO: 30 - DNA, 52 bp, Artificial Sequence
  Primer
acttcctccgccaccagaaccaccgcctcctttgatttccaccttggtcccc

SEQ ID NO: 31 - DNA, 67 bp, Artificial Sequence
  Primer
ggaggcggtggttctggtggcggaggaagtggcggcggaggatcccagatgcagctggtgcaatctg

SEQ ID NO: 32 - DNA, 97 bp, Artificial Sequence
  Primer
gtgatgccgcccatttcctcgttgccatcctcgagggatcctccgccgccacttcctccgccaccagaaccaccgcctccgacggtgaccgtggtcc

(19) United States
(12) Patent Application Publication
(10) Pub. No.: US 2024/0058380 A1
(43) Pub. Date: Feb. 22, 2024

(54) CORONA VIRUS-SPECIFIC T CELL RECEPTOR FUSION CONSTRUCTS, VECTORS ENCODING THE SAME, T CELLS COMPRISING THE SAME AND USES THEREOF

(71) Applicant: ALBERT-LUDWIGS-UNIVERSITÄT FREIBURG, Freiburg (DE)

(72) Inventors: Wolfgang SCHAMEL, Freiburg (DE); Ira GODBOLE, Breisach am Rhein (DE); Sascha YOUSEFI, Gundelfingen (DE); Kevin CIMINSKI, Freiburg (DE); Martin SCHWEMMLE, Freiburg (DE)

(21) Appl. No.: 18/032,101

(22) PCT Filed: Oct. 22, 2020
(86) PCT No.: PCT/EP2021/079307

§ 371 Date: Apr. 14, 2023

(30) Foreign Application Priority Data
Oct. 22, 2020 (EP) 20203227.2
Jun. 24, 2021 (EP) 21181578.2

(51) Int. Cl. A61K 35/17; C07K 14/725; C07K 16/10; C07K 16/40; C12N 5/0783; C12N 15/86; A61K 39/00; A61P 31/14

(52) U.S. Cl. CPC A61K 35/17; C07K 14/7051; C07K 16/1003; C07K 16/40; C12N 5/0636; C12N 15/86; A61K 39/4611; A61K 39/4632; A61K 39/464838; A61P 31/14; A61K 2239/22; A61K 2239/21

(57) ABSTRACT

The present invention is inter alma concerned with a T cell receptor fusion construct comprising two specific peptidic moieties, one of these two moieties binding to the spike protein from coro-naviruses and binding to ACE2, in particular the spike proteins from SARS-CoV-2 and/or SARS-CoV-1, and one of these moieties being a protein of the T cell receptor complex. A vector comprising the genetic information encoding the T cell receptor fusion construct is also part of the present invention, as well as a process of transfecting or transducing T cells and a modified T cell comprising the T cell receptor fusion construct. Importantly, the present invention also relates to a T cell receptor fusion construct, a vector or a modified T cell for use in the treatment of a disease and in particular for use in the treatment of a disease caused by a coronavirus such as e.g. COVID-19 or SARS.

### FIELD OF THE INVENTION

The present invention is in the field of treatment of diseases caused by coronaviruses, in particular COVID-19 and SARS treatment. The present invention relates to a T cell receptor fusion construct comprising a peptidic moiety binding to the spike protein from a coronavirus, wherein the spike protein binds to ΔCE2, and a peptidic moiety of the T cell receptor complex as defined herein. For example, the first moiety binds to the spike protein from SARS-CoV-2 (causing COVID-19), from SARS-CoV-1 (causing the SARS disease) and/or from other coronaviruses using ACE2 as their receptor to enter host cells. A vector comprising the genetic information encoding the T cell receptor fusion construct is also part of the present invention. The present invention is also concerned with a process of transfecting or transducing T cells and a modified T cell comprising the T cell receptor fusion construct. Finally, the present invention relates to a T cell receptor fusion construct, a vector or a modified T cell for use in the treatment of a disease, in particular for use in the treatment of coronavirus diseases including, but not limited to, COVID-19 or SARS.

### BACKGROUND OF THE INVENTION

The coronavirus disease 2019 (COVID-19) pandemic has hit most, if not all, countries and has led to more than 1 million death cases world-wide—with 220 000 deaths in the U.S.A. (as of Oct. 19, 2020, John Hopkins University). The disease is caused by a new virus of the beta-coronavirus family, the severe acute respiratory syndrome coronavirus 2 (SARS-CoV-2). Neither a vaccination nor an effective treatment is available to date (as of October 2020), wherein—as of June 2021 several vaccines have meanwhile been approved while effective treatments are still lacking. Accordingly, there is inter alia an immense need for an effective treatment of COVID-19.

A very related virus, the severe acute respiratory syndrome coronavirus 1 (SARS-CoV-1), has caused a much smaller disease outbreak in China in 2002 and 2003 with 800 deaths, called the severe acute respiratory syndrome (SARS) disease. Also for this disease neither a vaccination nor a good treatment is available. Accordingly, there is also still the need for an effective treatment of the SARS disease.

On a general level, there is still the need for effective treatments of diseases caused by coronaviruses.

### OBJECTS AND SUMMARY OF THE INVENTION

The inventors of the present invention have surprisingly found that the underlying concept of a successful immunocancer therapy can also be used to treat COVID-19 caused by SARS-CoV-2 and the SARS disease caused by SARS-CoV-1, and more generally diseases caused by coronaviruses that comprise a spike protein (S protein), wherein the S protein binds to the host protein angiotensin converting enzyme 2 (ACE2). In essence, the inventors used the concept of T cell re ceptor fusion constructs in cancer therapy, which make the T cells recognize cancer cells, and adapted it such that the T cells recognize coronavirus-infected cells, in particular SARS-CoV-2-infected and SARS-CoV-1-infected cells instead of cancer cells. More particularly, the T cell receptor (TCR) complex comprising an engineered T cell receptor fusion construct according to the present invention (and thus also the T cells expressing the same) recognizes the S protein characterized in that it binds to ACE2, in particular the S protein of SARS-CoV-2 and SARS-CoV-1 on the surface of infected cells.

In the field of cancer treatment, the use of chimeric antigen receptors (CARs) in immunocancer therapy has revolutionized the treatment of haematological tumours (June and Sadelain, 2018) and two different CARs were approved by the FDA in 2018 (Boyer, 2018). CARs consist of a do main that binds to the tumour antigen (in most cases an antibody-derived single chain Fv fragment), a linker and transmembrane region and the cytoplasmic tail of the TCR's ζ subunit and the tail of the co-stimulatory receptors CD28 or 4-1BB (FIG. 1) (Sadelain, 2016; Schamel and Reth, 2012). When expressed in the tumour patient's T cells, these cells are reprogrammed to recognize the tumour. After infusion of the modified T cells into the patient, the cells recognize and kill the cancer. However, CARs do not use the full TCR machinery and the treatment is only successful in haematological, but not in solid tumours.

T cell receptor fusion constructs (TRuCs) also consist of a tumour-binding domain and thus re program T cells to recognize the tumour. However, in stark contrast to the CARs, this domain is fused to the complete TCR (FIG. 1) (Baeuerle et al., 2019; Hardy et al., 2020). Thus, TRuCs use the full signalling potential of the TCR. In case of the εTRuC the binding domain is fused to the TCR's CD3c chain. In pre-clinical mouse models of haematological cancers, the εTRuC-T cells killed the cancer faster and better than the CAR-T cells (Baeuerle et al., 2019). Importantly, the εTRuC-T cells were also effective against solid tumours in mice and in human patients—situations where CAR-T cells fail (information from www.tcr2.com). So far TRuCs have only been used to combat cancer cells but the inventors surprisingly found that TRuCs can also be used to combat COVID19 and the SARS disease, namely by directing the TRuCs to the S protein of the viruses.

Thus, to enter into their host cells, SARS-CoV-2 and SARS-CoV-1 use their S protein that is pre sent on the virus envelope and binds to the host ACE-2 (FIG. 2) (Van et al., 2020; Zhou et al., 2020). In cells infected with the SARS-CoV-2 or SARS-CoV-1 virus, the S protein is present on the cell surface (Walls et al., 2020), thus the cells infected with the virus can be distinguished extracellularly from healthy cells.

The inventors engineered TRuCs, exemplified by εTRuCs, that recognize the infected cells instead of cancer cells, and the inventors inter alia showed that cells expressing these εTRuCs are able to kill cells with the SARS-CoV-2 S protein. Without wishing to be bound to a theory, it is strongly assumed that the TRuCs according to the present invention and the resulting TCR complexes in T cells are superior over other chimeric treatment options (including CARs) because the fully assembled TCR complexes are much more sensitive (Gudipati et al., 2020), i.e. they recognize coronavirus-infected cells, in particular SARS-CoV-2-infected cells and SARS-CoV-1-infected cells, with S protein on their surface already at a very early stage of the lifecycle of the virus, i.e. already when minor amounts of S protein are present on the cell surface. These cells are then eliminated. In other words, the infected cells are attacked and killed before they can enter a late stage of the lifecycle of the virus, where much higher amounts of the S protein are present on the cell surfaces, and where the virus still has a chance to multiply and infect further cells. There is reason to believe that other chimeric treatment options (including CARs) do not exhibit such a level of sensitivity such that they might recognize SARS-CoV-2- and SARS-CoV-1-infected cells at a stage, which might already be late or too late in terms of the lifecycle of the virus in a given cell.

Furthermore, the inventors inter alia found that the treatment of SARS-CoV-2 infected cells with their engineered T cells did not lead to massive cytotoxicity towards the infected cells (which would provide a significant beneficial effect in therapy, in particular when considering that in severe COVID-19 cases a large percentage of the lung cells is infected, and it would be detrimental to kill such a large amount of lung cells but much more preferred to heal these cells), but resulted in a complete rescue of the translational shutdown despite ongoing viral replication. The data of the present application show that engineered TRuC T cell products can be used in therapy against SARS-CoV-2, most likely by exposing infected cells to the host innate immune sys tem.

It should be noted by way of background in this respect that one of the major innate antiviral de fence mechanisms is the type I interferon (IFN-I) system, the induction of which stimulates the expression of a large number of interferon-stimulated genes (ISGs). In case of coronaviruses the viral RNA is sensed by retinoic acid-inducible gene I (RIG-I), thereby activating innate immune signaling (Hu et al., 2017; Sparrer and Gack, 2015). However, these viruses evade the innate immune response by abrogating host mRNA translation, thus preventing expression of IFN-I and ISGs (Hsu et al., 2021; Kamitani et al., 2009; Lei et al., 2020; Narayanan et al., 2008; Puray-Chavez et al., 2020; Thoms et al., 2020; Xia H et al., 2020; Yuen et al., 2020). In SARS-CoV-1 and SARS-CoV-2 multiple viral proteins are responsible for this translational shutdown (Hsu et al., 2021; Kamitani et al., 2009; Narayanan et al., 2008; Puray-Chavez et al., 2020; Thoms et al., 2020). For example, the non-structural protein 1 (Nsp1) inhibits nuclear export of host mRNA, but also blocks ribosomal translation by binding to the 40S ribosomal subunit (Kamitani et al., 2009; Narayanan et al., 2008; Thoms et al., 2020; Zhang et al., 2021) (Lapointe et al., 2021; Yuan et al., 2020b). In addition, Nsp5, Nsp14 and Nsp15 can also halt translation of host mRNA (Hsu et al., 2021; Lei et al., 2020; Xia H et al., 2020; Yuen et al., 2020). Indeed, SARS-CoV-2 infection as well as overexpression of these Nsps in Vero E6 or HEK293T cells inhibits the production of IFN-I and induction of ISGs (Lei et al., 2020; Puray-Chavez et al., 2020; Thorns et al., 2020; Xia H et al., 2020; Yuen et al., 2020). However, viral proteins can still be produced, most likely due to a special feature of the 5′ untranslated region of viral mRNAs (Tanaka et al., 2012).

Overall and again without wishing to be bound to a theory, it is strongly assumed that, upon recognition of infected cells, the S-specific εTRuC T cells are activated (as seen by the killing in the first hours) and secrete cytokines or other factors that can rescue the infected cells from the protein synthesis shutdown.

In the first aspect, the present invention is directed to a T cell receptor fusion construct comprising (a) a peptidic moiety binding to a spike protein, wherein the spike protein is characterized in that (i) it is from a coronavirus and (ii) it binds to ACE2; and (b) a peptidic moiety selected from the group consisting of T cell receptor α chain, T cell receptor β chain, CD3γ, CD3ε, CD3δ, and variants of any of the foregoing. The ACE2, the spike protein is binding to, is preferably human ACE2.

In an embodiment thereof, the T cell receptor fusion construct comprises (a) a peptidic moiety binding to the spike protein from SARS-CoV-2, SARS-CoV-1 and/or other coronaviruses; and (b) a peptidic moiety selected from the group consisting of T cell receptor α chain, T cell receptor β chain, CD3γ, CD3ε, CD3δ, and variants of any of the foregoing.

In another embodiment thereof, the T cell receptor fusion construct comprises (a) a peptidic moiety binding to the spike protein from SARS-CoV-2 and/or SARS-CoV-1; and (b) a peptidic moiety selected from the group consisting of T cell receptor α chain, T cell receptor β chain, CD3γ, CD3ε, CD3δ, and variants of any of the foregoing.

In a preferred embodiment, the T cell receptor α chain, T cell receptor β chain, CD3γ, CD3ε, and CD36 are human T cell receptor α chain, human T cell receptor β chain, human CD3γ, human CD3ε, and human CD3δ.

In a preferred embodiment, the T cell receptor fusion construct further comprises a peptidic linker between the peptidic moiety (a) and the peptidic moiety (b). The purpose of the linker is to separate the peptidic moiety (a) from the peptidic moiety (b) such that the peptidic moiety (a) is in a suitable steric position and distance from peptidic moiety (b) and the cell membrane. It can be preferred that this peptidic linker is a linker comprising at most about 20 amino acids. It is particularly preferred that the peptidic linker is selected from the group consisting of polyglycine, G4S (SEQ ID NO: 7), (G4S)×2, (G4S)×3, (G4S)×4, GGSG (SEQ ID NO:10), GSTSGSGKPGSGEGSTKG (SEQ ID NO:11) and LDGSGGDV (SEQ ID NO:12). Some of these linkers have been successfully used in the present invention, namely (G4S) and (G4S)×3, and others have been successfully used in related setups, see (Baeuerle et al., 2019; Yousefi et al., 2019).

In a preferred embodiment, the T cell receptor fusion construct according to the first aspect further comprises a signal peptide before the peptidic moiety (a), wherein it is preferred that this signal peptide is located at the very N-terminus of the T cell receptor fusion construct. A signal peptide can be selected from the group consisting of the ACE2 signal peptide (SEQ ID NO: 1), the CR3022 light chain signal peptide (SEQ ID NO: 4), the CR3022 heavy chain signal peptide (SEQ ID NO:13), the Ig-α chain signal peptide (SEQ ID NO:14), the CD8α signal peptide (SEQ ID NO:15), GM-CSF signal peptide (SEQ ID NO:16), T cell receptor α chain signal peptide T1 (SEQ ID NO:17), T cell receptor α chain signal peptide OT1 (SEQ ID NO:18), T cell receptor β chain signal peptide T1 (SEQ ID NO:19), T cell receptor β chain signal peptide OT1 (SEQ ID NO:20), T cell receptor γ signal peptide (SEQ ID NO:21), T cell receptor δ signal peptide (SEQ ID NO:22) and HLA-A signal peptide (SEQ ID NO:23). When it comes to two particularly preferred embodiments relating to peptidic moiety (a) of the first aspect of the present invention, namely ACE2 and the CR3022 scFv, any signal peptide can in principle be used but it can be preferred to use their corresponding signal peptides, i.e. the ACE2 signal peptide (SEQ ID NO: 1) for ACE2 and the CR3022 light chain signal peptide (SEQ ID NO: 4) for the CR3022 scFv, as it is shown in FIG. 3.

In a preferred embodiment, the peptidic moiety (b) comprises an extracellular region, a transmembrane region and an intracellular region. Thus, preferably due to the activity of the signal peptide, the T cell receptor fusion construct is targeted to the secretory pathway and, due to the transmembrane region, bound to the cell membrane, and is present there in an orientation such that the N-terminal peptidic moiety (a) is completely present in the extracellular domain, whereas the peptidic moiety (b) that includes the transmembrane regions is not only present extracellularly but also spans through the membrane and has an intracellular region, as mentioned above. This ensures (i) a correct localization of the T cell receptor fusion construct, (ii) a correct assembly with the remaining TCR subunits, in order to arrive at a functional TCR complex, and (iii) a correct downstream, i.e. intracellular, signaling.

In a preferred embodiment, the peptidic moiety (a) is selected from the group consisting of an antibody-derived fragment binding to the spike protein, preferably the spike protein from SARS-CoV-2 and/or SARS-CoV-1; an aptamer binding to the spike protein, preferably the spike protein from SARS-CoV-2 and/or SARS-CoV-1; and a receptor binding to the spike protein, preferably the spike protein from SARS-CoV-2 and/or SARS-CoV-1, or a binding domain thereof (wherein the binding domain of the receptor binds to the spike protein, preferably the spike protein from SARS-CoV-2 and/or SARS-CoV-1).

In a particularly preferred embodiment, the peptidic moiety (a) is a receptor binding to the spike protein, preferably to the spike protein from SARS-CoV-2 and/or SARS-CoV-1, or a binding do main thereof. It is most preferred that the receptor is ACE2, which binds to the spike protein, preferably to the spike proteins from SARS-CoV-2 and SARS-CoV-1, or a spike protein-binding domain thereof. It is furthermore especially preferred that the ACE2 is human ACE2. It is particularly preferred that the ACE2 lacks the ACE2-ACE2 dimerization motif. It can be preferred that the binding domain of ACE2 comprises the amino acid sequence of SEQ ID NO: 2 or a sequence that is at least 85% identical thereto. In a particular embodiment, the peptidic moiety (a) has the amino acid sequence of SEQ ID NO: 2 or a sequence that is at least about 7096, at least about 80&, at least about 85%, at least about 88%, at least about 90%, at least about 92%, at least about 95% or at least about 98% identical thereto.

In a particular preferred embodiment, the peptidic moiety (a) is an antibody-derived fragment and the antibody-derived fragment is a single chain fragment (scFv) or a single domain fragment, as in cameloid antibodies. It can be especially preferred that the single chain fragment comprises the amino acid sequence of SEQ ID NO: 5 and the amino acid sequence of SEQ ID NO: 6. Furthermore, it can be especially preferred that the single chain fragment (scFv) further comprises a peptidic linker between the amino acid sequences of SEQ ID NO: 5 and SEQ ID NO: 6. The purpose of the linker is to separate the amino acid sequences of SEQ ID NO: 5 and SEQ ID NO: 6 such that the two sequences are able to properly fold into an antigen binding fragment. It can be preferred that this peptidic linker is a linker comprising at most about 25 amino acids. It is particularly preferred that the peptidic linker is selected from the group consisting of (G4S)×3, (G4S)×4, (G4S)×5, PNGASQSSSASHTGSAPGS (SEQ ID NO:24), and RPLSYRPPFPFGFPSVRP (SEQ ID NO:25). A (G4S)×3 linker can be especially preferred for the afore-mentioned scFv, in particular the CR3022 scFv.

In a preferred embodiment, the spike protein is characterized in that it is from a coronavirus selected from the group consisting of SARS-CoV-2, SARS-CoV-1 and HCoV-NL63 (whose spike proteins bind to ACE2).

In another preferred embodiment, the spike protein is characterized in that it is from SARS-CoV-1 (whose spike protein binds to ACE2). In another preferred embodiment, the spike protein is characterized in that it is from HCoV-NL63 (whose spike protein binds to ACE2).

In the most preferred embodiment, the spike protein is characterized in that it is from SARS-CoV-2 (whose spike protein binds to ACE2).

In the second aspect, the present invention is directed to a vector comprising the genetic information encoding a T cell receptor fusion construct according to the first aspect. Of course, any embodiment of the first aspect is also included in the second aspect.

In a preferred embodiment, the vector is selected from the group consisting of a lentiviral vector, a DNA vector, an RNA vector, a plasmid vector, a cosmid vector, a herpes virus vector, a measles virus vector, an adenoviral vector and a retrovirus. It can be preferred that the RNA vector is an mRNA vector. Particularly preferred for the present invention is a lentiviral vector or an adenoviral vector.

In a preferred embodiment, the vector of the second aspect further comprises a promoter operably linked to the genetic information encoding a T cell receptor fusion construct. It can be preferred that the promoter is selected from the group consisting of EF1α, β-actin, TGK, a combination of the long terminal repeat of HTLV and EF1α, CMV and SFFV.

In the third aspect, the present invention is directed to a process of transfecting or transducing T cells with a vector according to the second aspect of the present invention, wherein the T cells are collected and cultivated ex vivo, thereafter transfected or transduced with a vector according to the second aspect, and thereafter grown and expanded ex vivo. Of course, any embodiment of the second aspect is also included in the third aspect.

The steps and embodiments of the third aspect are the steps that are typically carried out when treating a patient using a therapy based on a CAR or a TRuC. Reference is made exemplary in this respect to the treatment of cancer patients by either CARs or TRuCs. As claimed in the third aspect, these steps are at least the step of collecting and cultivating T cells ex vivo, thereafter transfecting or transducing the T cells, and thereafter growing and expanding the T cells ex vivo.

In the fourth aspect, the present invention is directed to a modified T cell comprising a T cell receptor fusion construct according to the first aspect of the present invention. Of course, any embodiment of the first aspect is also included in the second aspect.

In the fifth aspect, the present invention relates to the T cell receptor fusion construct according to the first aspect, or the vector according to the second aspect, or the modified T cell according to the fourth aspect for use in the treatment of a disease. In other words, the present invention relates to the T cell receptor fusion construct according to the first aspect, or the vector according to the second aspect, or the modified T cell according to the fourth aspect for use in a method of treatment of the human body by therapy. Of course, any embodiment of the afore mentioned aspects are also included in the fifth aspect.

In the sixth aspect, the present invention relates to the T cell receptor fusion construct according to the first aspect, or the vector according to the second aspect, or the modified T cell according to the fourth aspect for use in the treatment of a disease caused by a coronavirus, in particular the coronavirus disease 2019 (COVID-19) or the severe acute respiratory syndrome (SARS) disease or symptoms associated with a HCoV-NL63 infection. In other words, the present invention relates to the T cell receptor fusion construct according to the first aspect, or the vector according to the second aspect, or the modified T cell according to the fourth aspect for use in a method of treating a patient suffering from a disease caused by a coronavirus, in particular a patient suffering from the coronavirus disease 2019 (COVID-19) or a patient suffering from the severe acute respiratory syndrome (SARS) disease or a patient suffering from symptoms associated with a HCoV-NL63 infection. Of course, any embodiment of the afore-mentioned aspects are also included in the sixth aspect.

In an embodiment of the sixth aspect, a T cell receptor fusion construct according to the first aspect, wherein the spike protein is characterized in that it is from SARS-CoV-2, or a vector of the second aspect, wherein the spike protein is characterized in that it is from SARS-CoV-2, or a modified T cell according to the fourth aspect, wherein the spike protein is characterized in that it is from SARS-CoV-2, is for use in the treatment of the coronavirus 2019 (COVID-19) disease.

In another embodiment of the sixth aspect, a T cell receptor fusion construct according to the first aspect, wherein the spike protein is characterized in that it is from SARS-CoV-1, or a vector of the second aspect, wherein the spike protein is characterized in that it is from SARS-CoV-1, or a modified T cell according to the fourth aspect, wherein the spike protein is characterized in that it is from SARS-CoV-1, is for use in the treatment of severe acute respiratory syndrome (SARS) disease.

In yet another embodiment of the sixth aspect, a T cell receptor fusion construct according to the first aspect, wherein the spike protein is characterized in that it is from HCoV-NL63, or a vector of the second aspect, wherein the spike protein is characterized in that it is from HCoV-NL63, or a modified T cell according to the fourth aspect, wherein the spike protein is characterized in that it is from HCoV-NL63, is for use in the treatment of symptoms associated with a HCoV-NL63 infection.

In an embodiment, where the peptidic moiety (a) of the T cell receptor fusion construct binds to the spike protein from SARS-CoV-2 but fails to (substantially) bind to the spike protein from SARS-CoV-1, a corresponding T cell receptor fusion construct, or the corresponding vector ac cording to the second aspect, or the corresponding modified T cell according to the fourth aspect is for use in the treatment of coronavirus disease 2019 (COVID-19).

In an embodiment, where the peptidic moiety (a) of the T cell receptor fusion construct binds to the spike protein from SARS-CoV-1 but fails to (substantially) bind to the spike protein from SARS-CoV-2, a corresponding T cell receptor fusion construct, or the corresponding vector according to the second aspect, or the corresponding modified T cell according to the fourth aspect is for use in the treatment of the SARS disease.

In a particularly preferred embodiment of the sixth aspect, the patient suffering from coronavirus disease 2019 (COVID 19) or the severe acute respiratory syndrome (SARS) disease is selected from the group consisting of a patient with a severe course of these diseases, an immunodeficient patient (e.g. an HIV patient or a primary chronic immunodeficiency), a patient of the high-risk group related to COVID-19 or SARS, and a patient suffering from a chronic COVID-19 or SARS infection.

The sixth aspect of the present invention may alternatively be expressed as follows: A method of treating coronavirus disease 2019 (COVID 19) or the severe acute respiratory syndrome (SARS) disease in a subject in need thereof, comprising administering to the subject a therapeutically effective composition comprising the modified T cell according to the fourth aspect.

The present invention further relates to the following methods of treatments in the sixth aspect. In an embodiment, the present invention relates to a method of treating a disease caused by a coronavirus in a subject in need thereof, comprising administering to the subject a therapeutically effective composition comprising the modified T cell according to the fourth aspect.

In another embodiment, the present invention relates to a method of treating coronavirus dis ease 2019 (COVID 19) in a subject in need thereof, comprising administering to the subject a therapeutically effective composition comprising the modified T cell according to the fourth aspect, wherein the spike protein is characterized in that it is from SARS-CoV-2.

In another embodiment, the present invention relates to a method of treating the SARS disease in a subject in need thereof, comprising administering to the subject a therapeutically effective composition comprising the modified T cell according to the fourth aspect, wherein the spike protein is characterized in that it is from SARS-CoV-1.

In another embodiment, the present invention relates to a method of treating symptoms associated with a HCoV-NL63 infection in a subject in need thereof, comprising administering to the subject a therapeutically effective composition comprising the modified T cell according to the fourth aspect, wherein the spike protein is characterized in that it is from HCoV-NL63.

In yet another embodiment, the present invention relates to a method of killing cells infected with a coronavirus in a subject in need thereof, comprising administering to the subject a therapeutically effective composition comprising the modified T cell according to the fourth aspect.

In yet another embodiment, the present invention relates to a method of rescuing the translational shutdown in a cell of a subject in need thereof, wherein the translational shutdown is induced in the cell by a coronavirus infection, comprising administering to the subject a therapeutically effective composition comprising the modified T cell according to the fourth aspect.

In yet another embodiment, the present invention relates to a method of exposing cells infected with a coronavirus to the host innate immune system in a subject in need thereof, comprising ad ministering to the subject a therapeutically effective composition comprising the modified T cell according to the fourth aspect.

In yet another embodiment, the present invention relates to a method of killing cells infected with SARS-CoV-2 in a subject in need thereof, comprising administering to the subject a therapeutically effective composition comprising the modified T cell according to the fourth aspect, wherein the spike protein is characterized in that it is from SARS-CoV-2.

In yet another embodiment, the present invention relates to a method of rescuing the translational shutdown in a cell of a subject in need thereof, wherein the translational shutdown is induced in the cell by a SARS-CoV-2 infection, comprising administering to the subject a therapeutically effective composition comprising the modified T cell according to the fourth aspect, wherein the spike protein is characterized in that it is from SARS-CoV-2.

In yet another embodiment, the present invention relates to a method of exposing cells infected with SARS-CoV-2 to the host innate immune system in a subject in need thereof, comprising ad ministering to the subject a therapeutically effective composition comprising the modified T cell according to the fourth aspect, wherein the spike protein is characterized in that it is from SARS-CoV-2.

In yet another embodiment, the present invention relates to a method of killing cells infected with SARS-CoV-1 in a subject in need thereof, comprising administering to the subject a therapeutically effective composition comprising the modified T cell according to the fourth aspect, wherein the spike protein is characterized in that it is from SARS-CoV-1.

In yet another embodiment, the present invention relates to a method of rescuing the translational shutdown in a cell of a subject in need thereof, wherein the translational shutdown is induced in the cell by a SARS-CoV-1 infection, comprising administering to the subject a therapeutically effective composition comprising the modified T cell according to the fourth aspect, wherein the spike protein is characterized in that it is from SARS-CoV-1.

In yet another embodiment, the present invention relates to a method of exposing cells infected with SARS-CoV-1 to the host innate immune system in a subject in need thereof, comprising ad ministering to the subject a therapeutically effective composition comprising the modified T cell according to the fourth aspect, wherein the spike protein is characterized in that it is from SARS-CoV-1.

In yet another embodiment, the present invention relates to a method of killing cells infected with HCoV-NL63 in a subject in need thereof, comprising administering to the subject a therapeutically effective composition comprising the modified T cell according to the fourth aspect, wherein the spike protein is characterized in that it is from HCoV-NL63.

In yet another embodiment, the present invention relates to a method of rescuing the translational shutdown in a cell of a subject in need thereof, wherein the translational shutdown is induced in the cell by a HCoV-NL63 infection, comprising administering to the subject a therapeutically effective composition comprising the modified T cell according to the fourth aspect, wherein the spike protein is characterized in that it is from HCoV-NL63.

In yet another embodiment, the present invention relates to a method of exposing cells infected with HCoV-NL63 to the host innate immune system in a subject in need thereof, comprising ad ministering to the subject a therapeutically effective composition comprising the modified T cell according to the fourth aspect, wherein the spike protein is characterized in that it is from HCoV-NL63.

### DESCRIPTION OF THE FIGURES

FIG. 1—Schematics of a CAR and an ETRuC-containing TCR.

In contrast to the CAR, the ETRuC assembles to the full TCR and thus has the full signalling capability. In both shown cases the engineered receptors employ an antibody-derived single chain Fv fragment as the anti-tumour binding domain (VL and VH).

FIG. 2—Schematic Entry of SARS-CoV-2 into cells.

Two routes for the entry of the virus into host cells exist. In the “fusion with the plasma membrane” the S protein is deposited on the cell surface. Whether the S protein recycles from the endosomes to the surface in the “endocytosis” route is presently unclear but S protein is in any case present at the cell surface during the lifecycle of the virus because it is expressed inside the infected cells and exported to the cell surface. Figure modified from (Kupferschmidt and Cohen, 2020).

FIG. 3—Schematics of the new SARS-CoV-2 and SARS-CoV-1-recognizing εTRuCs.

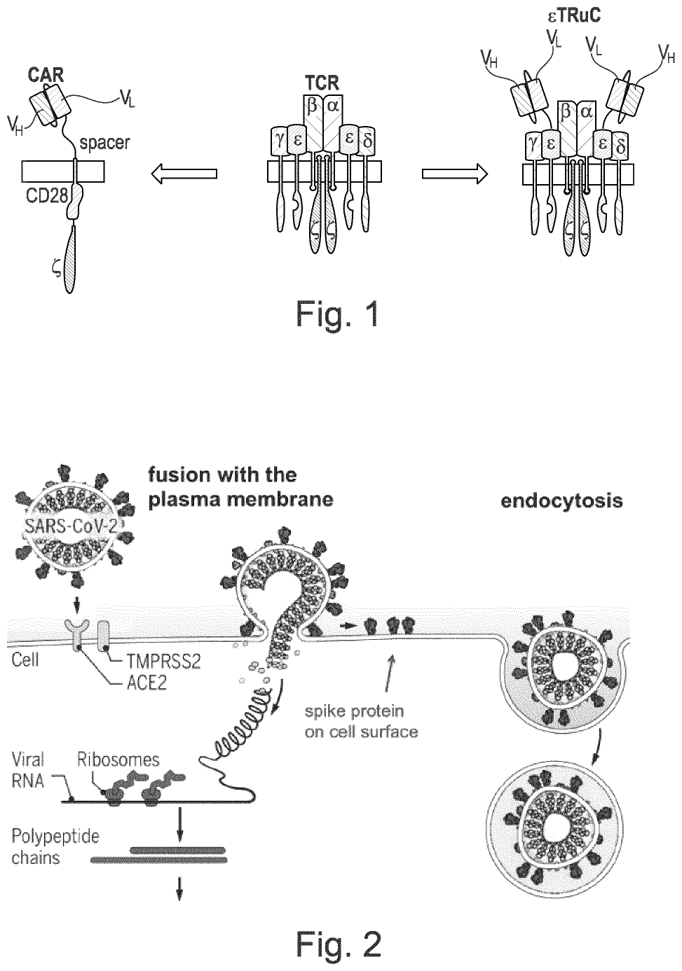

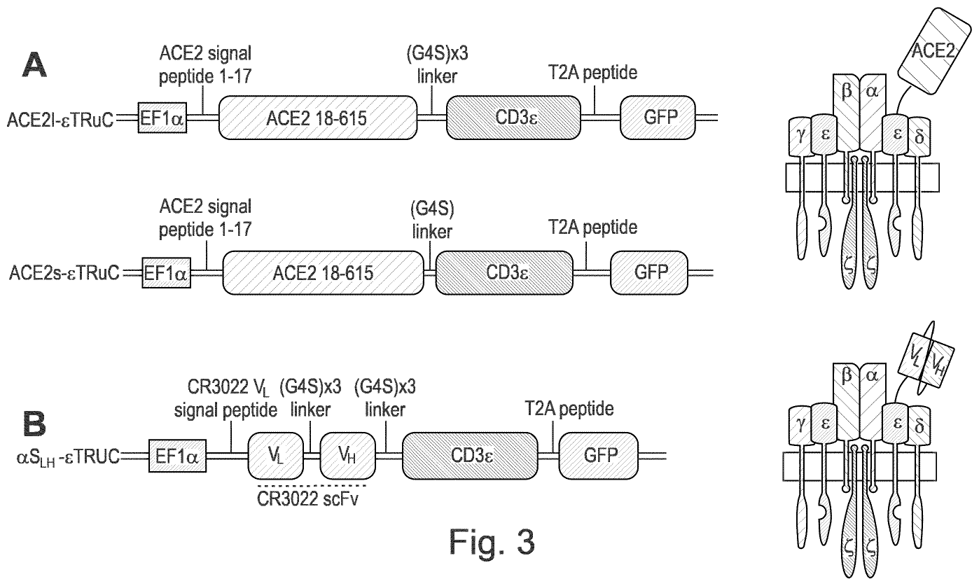

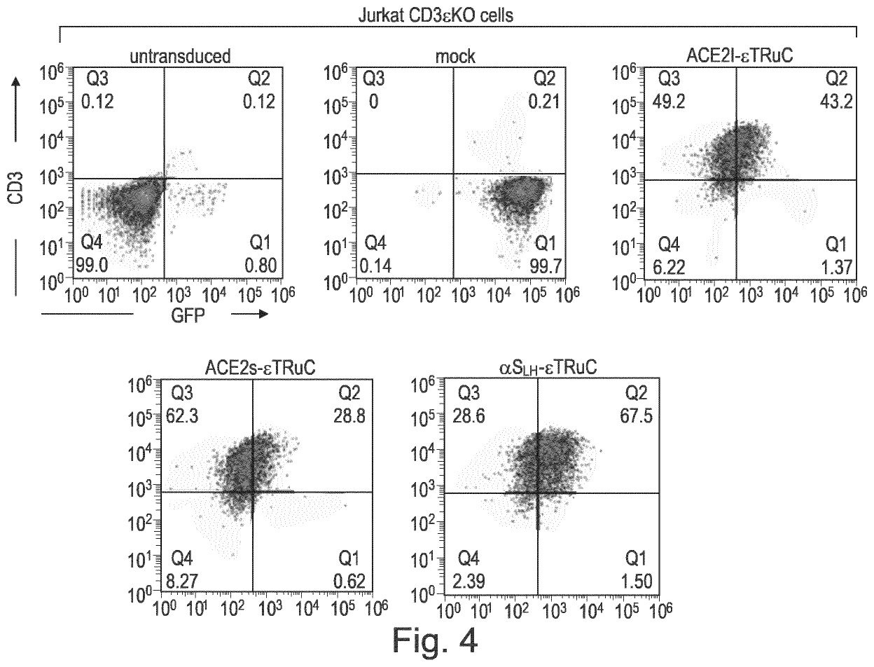

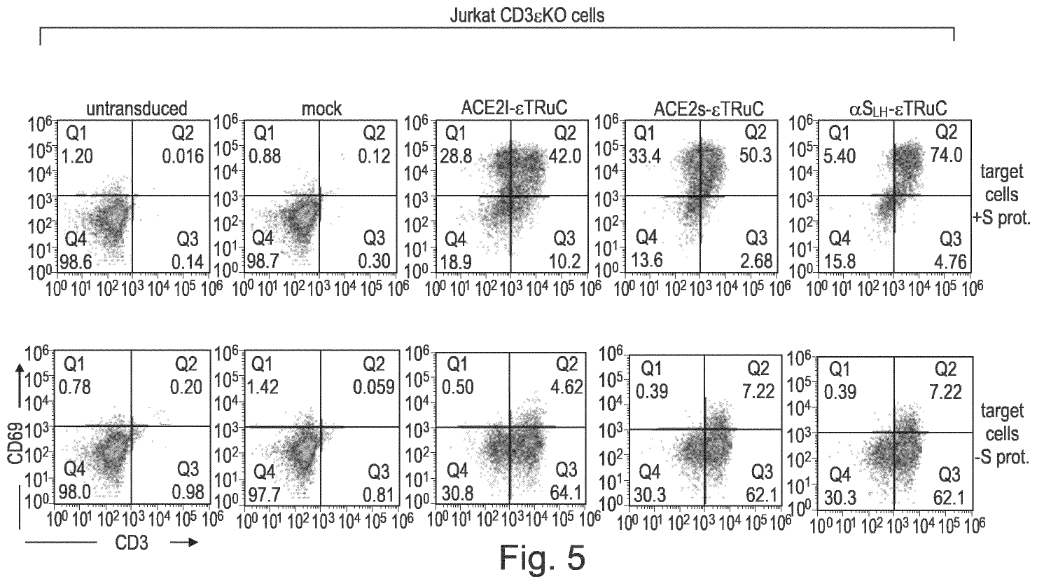

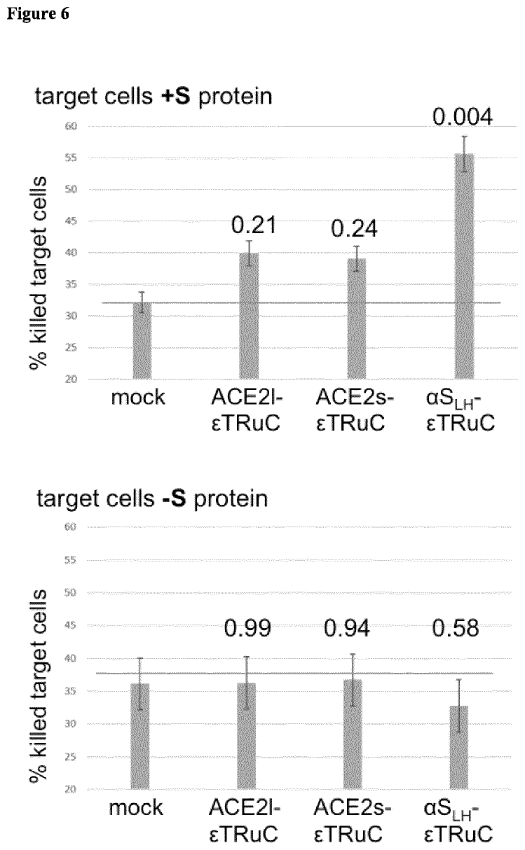

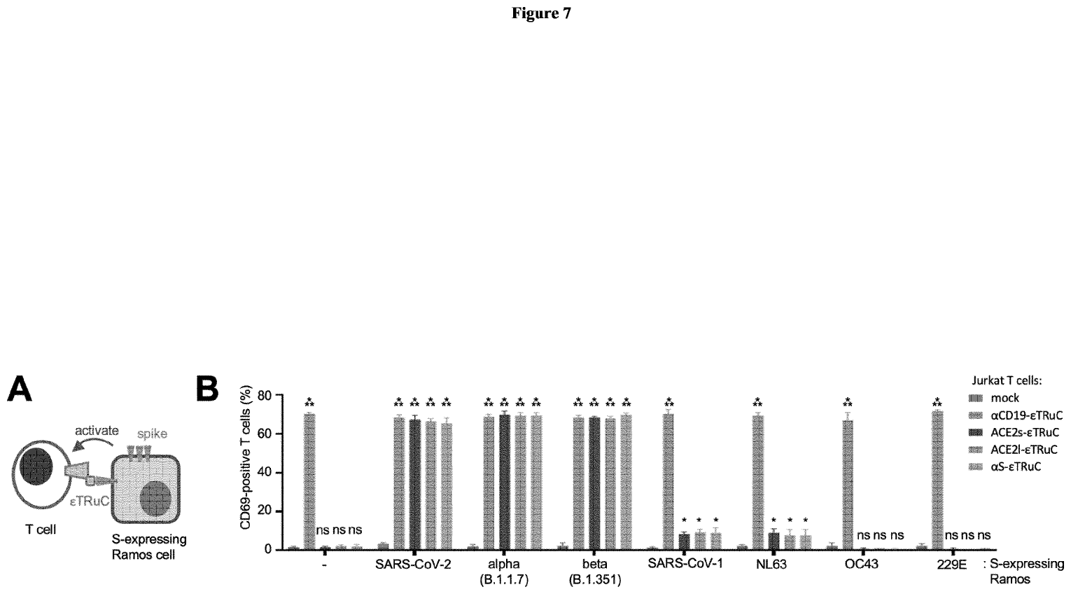

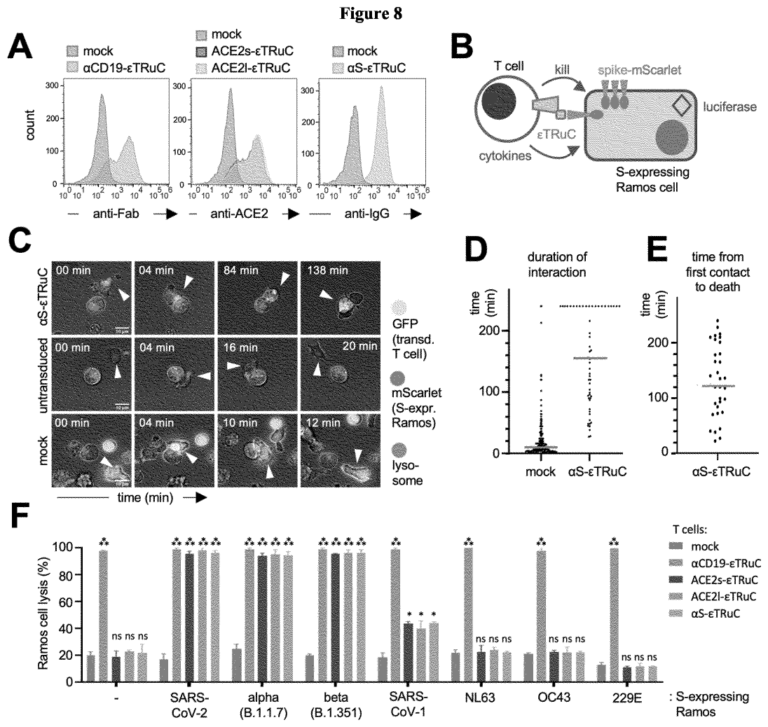

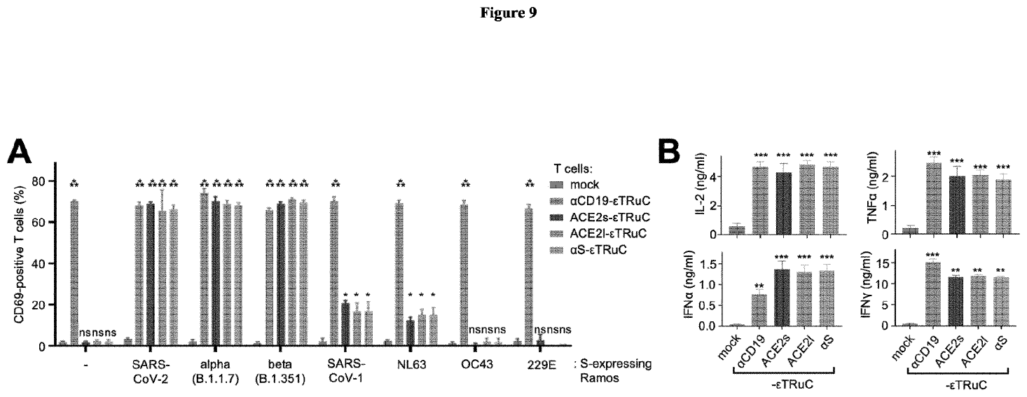

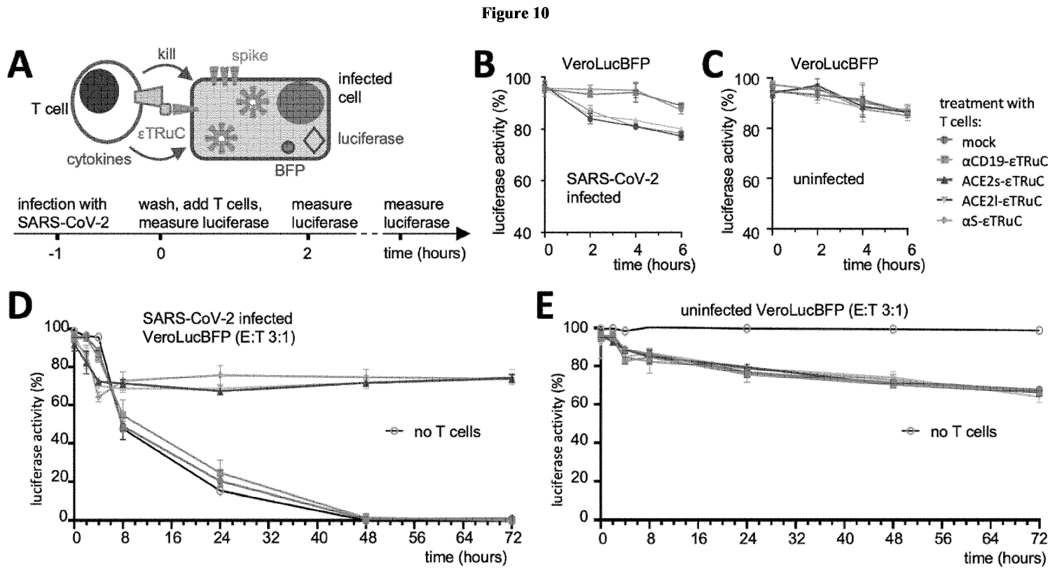

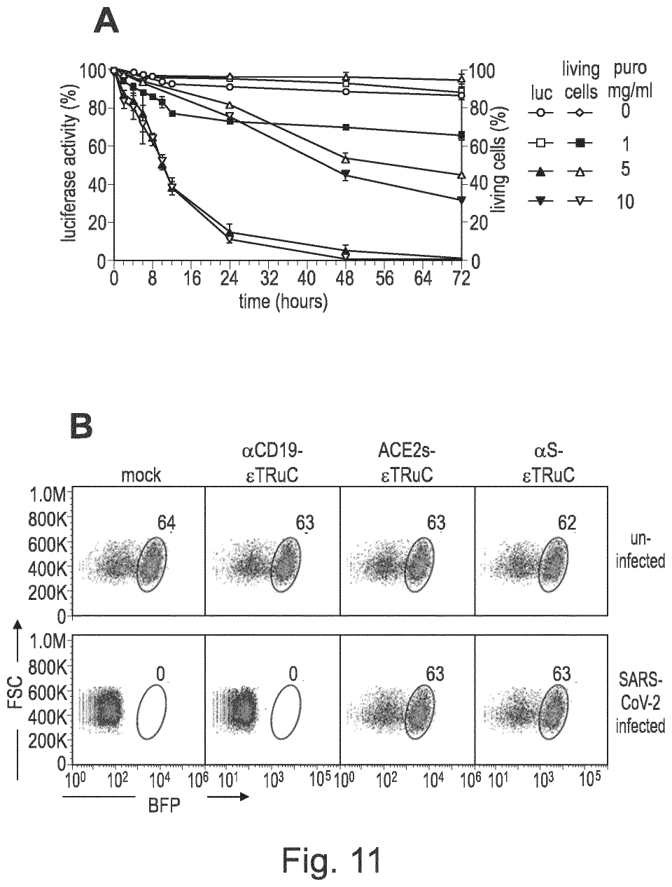

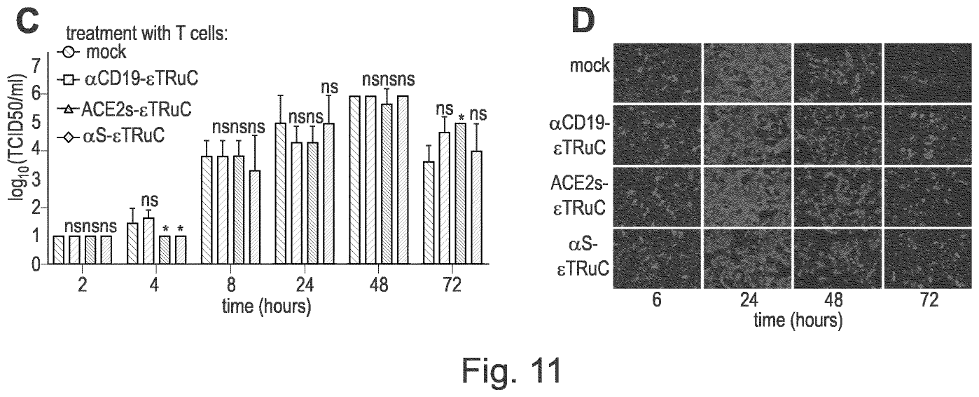

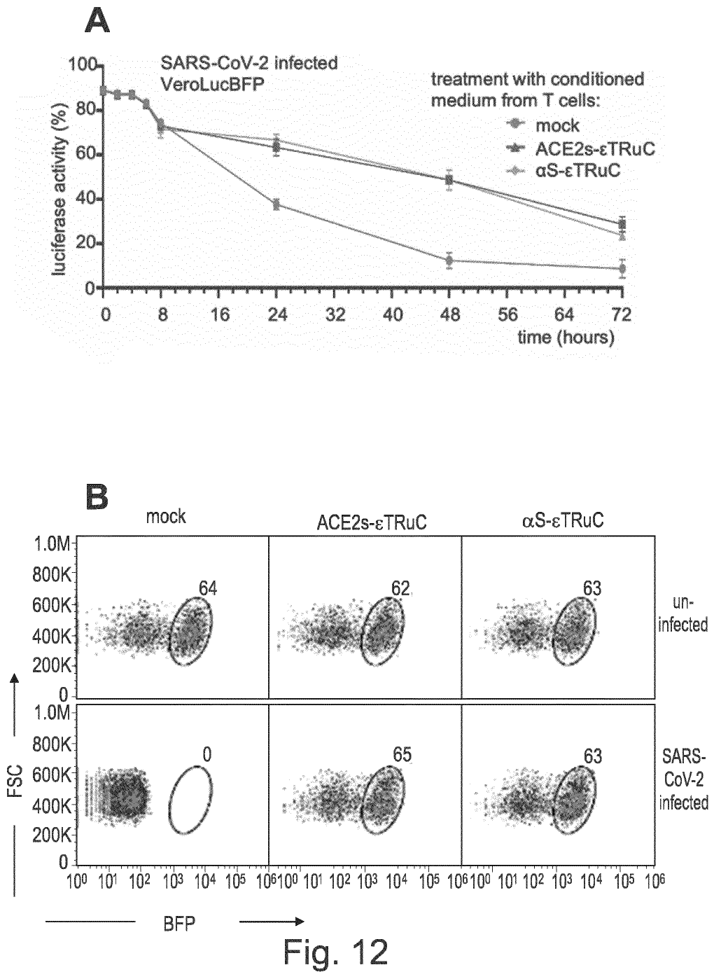

(A) the part of human ACE2 that binds to the S protein was fused to human CD3c using two different linker lengths. (B) a single chain Fv fragment of the anti-S protein antibody CR3022 was fused to human CD3c. All three TRuCs integrate into a full TCR complex (right panels). It was demonstrated using tumour-specific εTRuCs that some TCRs contain two εTRuCs as schematically shown in FIG. 1, and others contain one ETRuC and one endogenous CD3ε subunit as schematically shown in FIG. 3 (Baeuerle et al., 2019).

The sequences of the Peptide/protein elements depicted in FIG. 3 are as follows:

| Peptide/protein | Corresponding SEQ ID: |
|---|---|
| ACE2 signal peptide | SEQ ID NO: 1 |
| ACE2 18-615 | SEQ ID NO: 2 |
| CD3ε | SEQ ID NO: 3 |
| CR3022 signal peptide | SEQ ID NO: 4 |
| VL CR3022 scFv | SEQ ID NO: 5 |
| VH CR3022 scFv | SEQ ID NO: 6 |
| GGGGS (G4S) | SEQ ID NO: 7 |
| T2A peptide | SEQ ID NO: 8 |
| GFP | SEQ ID NO: 9 |

FIG. 4—Expression of the novel εTRuCs as part of a TCR on Jurkat CD3εKO cells.

Jurkat CD3εKO cells were left untransduced, transduced with a lentiviral vector encoding for only GFP (mock) or the SARS-CoV-2 and SARS-CoV-1-specific εTRuCs containing the T2A peptide GFP (right 3 panels). Transduced cells were sorted for the GFP+ cells and stained with fluorescent anti-CD3 antibodies. Fluorescence was quantified by flow cytometry.

FIG. 5—Activation of the εTRuC-expressing Jurkat cells by the SARS-CoV-2 S protein.

The Jurkat transductants from FIG. 4 were stimulated with target cells (Ramos cells) that either express the SARS-CoV-2 S protein (upper row) or not (lower row). After 9 hours the T cells were stained with anti-CD69 and anti-CD3 antibodies and measured by flow cytometry. Only the T cells that express a SARS-CoV-2 and SARS-CoV-1-specific εTRuC upregulated the activation marker CD69 upon co-culture with S protein expressing cells.

FIG. 6—Primary human T cells expressing the SARS-CoV-2 and SARS-CoV-1-specific εTRuCs can kill target cells that display the SARS-CoV-2 virus S protein.

Primary expanded human T cells were lentivirally transduced to express the new εTRuCs. The cells were then co-cultured for 6 hours with target cells (Ramos cells) that either express the SARS-CoV-2 S protein (upper row) or not (lower row). Then the percentage of dead target cells was measured using the luciferase-based killing assay in triplicates. Background unspecific killing by the mock transduced T cells is indicated by a blue line. In the presence of the S protein the new εTRuC-specific T cells killed the target cells better than the mock control. N=5 with two different donors.

FIG. 7—The new εTRuCs re-program Jurkat cells to recognize S-expressing cells.

A, By binding to the S-specific εTRuCs, Ramos cells expressing S activate the εTRuC Jurkat cells. B, Ramos cells without S and those expressing SARS-CoV-2 S were co-cultured with the Jurkat CD3εKO transductants from A and stained with anti-CD69 antibodies. After flow cytometric measurement, T cells were gated and the percentage of CD69-positive cells of triplicates is shown (the experiment was repeated more than 3 times, n>3).

FIG. 8—S-specific εTRuC T cells selectively eliminate S-expressing cells.

A, Primary human T cells were transduced with the lentiviral vectors encoding for the εTRuCs or the mock vector and expanded with IL-2. Surface expression of the εTRuCs was determined by anti-Fab, anti-ACE2 and anti-IgG staining as indicated. B, S-and luciferase-expressing Ramos cells are killed by the new εTRuC T cells. C, An αS-εTRuC T cell (green) with the lysosomes stained in pink and an S-mScarlet-expressing Ramos cell (red) were imaged and selected frames of the given times are shown (upper panel). Non-transduced (middle panel) and mock-transduced (green, lower panel) T cells were imaged together with the S-mScarlet-expressing Ramos cells (red). D, Quantification of the duration of interaction between S-mScarlet-expressing Ramos cells and αS-εTRuC or mock T cells from the 4 h videos. E, Quantification of the time it takes for an αS-εTRuC T cell to kill an S-mScarlet-expressing Ramos cell (the purple lines in D and E depict the median). F, Ramos cells expressing luciferase, BFP and the different S-proteins were co-cultured with the εTRuC T cells for 24 h at in a 1:1 ratio. Target cell lysis was measured by a loss of luciferase activity in triplicates (the experiment was repeated more than 3 times, n>3).

FIG. 9—S-specific εTRuC T cells are activated and secrete cytokines upon stimulation with 5 expressing cells.

A, Percent of CD69-positive εTRuC T cells after co-culture with the different S-expressing Ramos cells was determined by flow cytometry in triplicates. B, Secretion of cytokines by the εTRuC T cells following co-culture with the Ramos cells expressing SARS-CoV-2 S was quantified by ELISA in triplicates. A and B were repeated more than 3 times (n>3).

FIG. 10—SARS-CoV-2-infected cells are killed by S-specific εTRuC T cells.

A, The new εTRuC T cells recognise and kill VeroLucBFP cells infected with SARS-CoV-2 (upper panel); scheme of the infection and T cell treatment (lower panel). B and D, VeroLucBFP cells were infected with SARS-CoV-2 at an MOI of 0.05 for 1 h. After washing the εTRuC-transduced or mock T cells were added at a T: VeroLucBFP cell ratio of 3:1 (E:T). The luciferase activity was determined for 6 h (B) or 72 h (D). Samples without the addition of T cells were included in D. Triplicates are shown. C and E, The experiments were performed as in B and D, but the VeroLucBFP cells were not infected. B to E was done with three different T cell donors. B to E were repeated more than 3 times (n>3).

FIG. 11—εTRuC T cell treatment prevents the protein shutdown in VeroLucBFP cells, but does not limit viral replication.

A, VeroLucBFP cells were treated with puromycin (0-10 μg/ml). The luciferase activity (purple lines) and the percent of living cells (turquoise lines) were determined. B, VeroLucBFP cells were infected with SARS-CoV-2 (MOI 0.05) for 1 h or left uninfected. After washing the transduced T cells were added at a ratio of T: VeroLucBFP cell of 3:1 and at 72 h post infection the BFP-expression of the VeroLucBFP cells was quantified by flow cytometry. The percent of BFP+ cells is given. C and D, In an experiment as B, the viral titers in the supernatant of the infected and treated VeroLucBFP cells (C) and the amount of infected cells as seen by staining for the viral N protein in pink (D) was determined at several time points post infection. In D, the nuclei of the cells were stained with DAPI (blue).

FIG. 12—Soluble factors of the εTRuC T cells prevent the protein shutdown in VeroLucBFP cells.

A, Conditioned medium of mock, ACE2s- and αS-εTRuC T cells that were co-cultured with S-ex pressing Ramos cells was added to SARS-CoV-2-infected VeroLucBFP cells at 0 and again at 24 h post infection. Luciferase activity was measured in triplicates (the experiment was done twice, n=2). B, At 72 h post infection the BFP-expression of the VeroLucBFP cells treated with the conditioned medium was quantified by flow cytometry. The percent of BFP+ cells is given.

### DETAILED DESCRIPTION OF THE INVENTION

Before the present invention is described in more detail in the example section, the following definitions are introduced.

### 1. Definitions

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the pre sent invention, the term “consisting of” is considered to be a preferred embodiment of the term “comprising”. If hereinafter a group is defined to comprise at least a certain number of embodiments, this is also meant to encompass a group which preferably consists of these embodiments only.

A “T cell receptor fusion construct” as used herein is a protein. The moieties and elements de fined for a T cell receptor fusion construct as claimed herein, in particular moieties (a) and (b), i.e. the two different peptidic moieties of the T cell receptor fusion construct, are given in accordance with their numbering as moiety (a) before moiety (b) in the direction of the N-terminus to the C-terminus of the T cell receptor fusion construct. This means in terms of the amino acid sequence from the N-terminus to the C-terminus that moiety (a) comes first, followed by moiety (b). As the construct is defined in one embodiment to comprise the two moieties, this does, however, not mean that moiety (a) is necessarily found at the actual N-terminus of the T cell receptor fusion construct. It is evident from the description and the examples of the present application that a signal peptide is typically found at the very N-terminus before this signal peptide is cleaved off in order to arrive at the mature fusion construct that is found in a TCR complex at the cell mem brane. Furthermore, as is also evident from the description and the examples of the present application that a linker may be present in between moieties (a) and (b).

As is also evident from the examples of the present application, a further peptidic moiety may be fused to the T cell receptor fusion construct at the C-terminus, such as e.g. GFP or the like, wherein this further protein is typically fused via an optionally cleavable linker (an example for such a cleavable linker is the linker T2A as used herein). If helpful for detection and/or purification purposes (in particular for in vitro assays using the construct), a tag, such as e.g. a flag-tag, myc-tag or HA-tag, may be included close or at the N-terminus of the construct. However, such further peptidic moieties at the C-terminus (as e.g. GFP) or tags close to or at the N-terminus are typically not present in a construct that is used in a therapeutic manner to transfect or transduce T cells of a patient, in particular when treating e.g. COVID-19 or SARS.

The “T cell receptor fusion construct” of the present invention may also be referred to as “T cell receptor fusion construct that specifically binds a spike protein, wherein the spike protein is characterized in that (i) it is from a coronavirus and (ii) it binds to ACE2”. With respect to specific coronaviruses, e.g. SARS-CoV-2 and SARS-CoV-1, the term may also be referred to as “T cell receptor fusion construct that specifically binds the spike protein from SARS-CoV-2 and/or SARS-CoV-1”. The T cell receptor fusion construct of the present invention is capable of assembling—together with the respective further proteins—into a functional TCR complex, wherein “functional” means that the complex is properly folded, exported to the cell surface and capable of proper downstream signalling. The members of a TCR complex are CD3γ, CD3ε, T cell receptor α chain, T cell receptor β chain, CD3ζ, and CD3δ (see also FIG. 1 and FIG. 3 A and B, right side), and if reference is made herein to one of the proteins of the TCR complex, such as e.g. to “CD3ε”, this is meant to include all proteins that are regarded in the field as such a protein, even if there might be slight variations in the protein sequence. This thus also applies to the terms “T cell receptor α chain” and “T cell receptor β chain”, i.e. these terms include all proteins that are regarded in the field as such proteins, in particular because there is no unique protein sequence for the T cell receptor α chain or the T cell receptor β chain in view of their function as variable immune receptor with variable regions (based on differences in the amino acid sequences).

If the T cell receptor fusion construct is e.g. a construct comprising as (b) CD3γ, this construct would assemble with the remaining proteins CD3ε, T cell receptor α chain, T cell receptor β chain, CD3ζ and CD3δ into a functional T cell receptor complex. If the T cell receptor fusion construct is e.g. a construct comprising as (b) T cell receptor α chain, this construct would assemble with the remaining proteins CD3γ, CD3ε, T cell receptor β chain, CD3ζ and CD3δ into a functional T cell receptor complex.

The term “a variant of T cell receptor α chain, T cell receptor β chain, CD3γ, CD3ε or CD3δ” as used herein refers to a variant of any one of the foregoing proteins that is capable of (i) assembling into a functional T cell receptor complex and (ii) contributing to downstream signalling. An example of a “variant” as used herein is shown in FIG. 19 of WO 2020/193506, where a modified CD3E is depicted that comprises 2 RK-motifs in the cytoplasmatic, signalling region compared to only one RK motif in the signalling region of a “wt” CD3E as shown in FIG. 18 of WO 2020/193506 (disregarding for both situations the fused scFv moiety at the N-terminus as shown in FIGS. 18 and 19 of WO 2020/193506—this moiety corresponds to peptidic moiety (a) and not (b), where reference is made to a variant). The “CD3E variant” as shown in FIG. 19 of WO 2020/193506 is capable of assembling into a functional T cell receptor complex and additionally provides a strong downstream signalling, which is—compared to the “wt” situation—even more efficient.

The term “G4S” as used herein refers to a sequence of 5 amino acids, namely four glycines followed by a serine. This sequence of 5 amino acids is often used as linker between different protein moieties. It can be only a single “G4S”, i.e. only the 5 afore-mentioned amino acids, or the motif can be reiterated, e.g. twice or three times or four times or five times. This is indicated herein by “(G4S)×2” (twice), “(G4S)×3 (three times), “(GS)×4” (four times) and “(G4S)×5” (five times).

The term “signal peptide” as used herein refers to a signal peptide that is capable of targeting the construct to the secretory pathway where proteins are exported to the cell membrane. Since the peptidic moiety (b) comprises a transmembrane region, the construct is membrane-bound, with the peptidic moiety (a) being located exclusively in the extracellular space while the peptidic moiety (b) being located with its N-terminal region in the extracellular space followed by its afore-mentioned transmembrane region and a C-terminal intracellular signaling region. A vector comprising the genetic information encoding the T cell receptor fusion construct according to the first aspect will necessarily encode a T cell receptor fusion construct comprising a signal pep tide in order to ensure that the protein expressed therefrom is correctly targeted to the secretory pathway.

The term “an antibody-derived fragment binding to a spike protein, wherein the spike protein is characterized in that (i) it is from a coronavirus and (ii) it binds to ACE2” (such as the specific “antibody-derived fragment binding the spike protein from SARS-CoV-2 and/or from SARS-CoV-1”) as used herein refers to a typical antigen binding structure found in antibodies, in particular to the antigen binding fragment comprised of domains of the light chain (VL) and the heavy chain (VH), or a single domain in case of cameloid antibodies, such as those from alpaca. If the anti body sequence of an antibody (or a binding fragment thereof) binding to a spike protein, wherein the spike protein is characterized in that (i) it is from a coronavirus and (ii) it binds to ACE2, in particular the spike protein from SARS-CoV-2 or from SARS-CoV-1, is known, its antigen binding fragment or region is immediately evident. A “single chain fragment” or “scFv” as referred to herein is a linear fusion of the variable region of an antibody resulting in an antigen binding fragment made up from domains of the light chain (VL) and the heavy chain (VH). Such linear fusions are preferably humanized scFv structures. Exemplified in the present application is an scFv derived from the anti-S protein antibody CR3022 mentioned above, where the respective domains of the light chain (VL) and the heavy chain (VH) were taken and fused, using a (G4S)×3 linker in between. Once further suitable antibodies binding to a spike protein, wherein the spike protein is characterized in that (i) it is from a coronavirus and (ii) it binds to ACE2 (such as e.g. the S protein from SARS-CoV-2 or SARS-CoV-1) have been identified and sequenced, corresponding scFv can be generated accordingly and used in the present invention.

The term “ACE-2” as used herein is the abbreviation of “angiotensin converting enzyme 2”. Synonyms for ACE-2 are ACE-related carboxypeptidase, angiotensin-converting enzyme homolog (ACEH) and metalloprotease MPROT15. The S protein from SARS-CoV-2 and SARS-CoV-1 binds to ACE-2 (Van et al., 2020; Zhou et al., 2020), and also S proteins from other coronaviruses bind to ACE-2 (such as e.g. the S-protein from HCoV-NL63).

The term “CR3022” as used herein refers to an antibody that binds to the S protein from SARS-CoV-1 (Yuan et al., 2020) and to the S protein from SARS-CoV-2 (Wrapp et al., 2020). A single chain fragment derived from the sequences of CR3022 may comprise as S protein binding fragment the amino acid sequence of SEQ ID NO: 5 and the amino acid sequence of SEQ ID NO: 6, wherein such a single chain fragment is referred to herein as “CR3022 scFv”.

The term “spike protein” or “S protein” as used herein refers to the spike protein that is present on the virus envelope of coronaviruses and that is used by coronaviruses (such as SARS-CoV-2 and SARS-CoV-1) to enter into their host cells. The term as used herein includes all (mutant) versions of spike proteins, including e.g. the versions from the SARS-CoV-2 variants alpha (B.1.1.7) and beta (B.1.351). Accordingly, when reference is made herein to a “coronavirus”, this is meant to include all variants thereof, e.g. variants of SARS-CoV-2, SARS-CoV-1 and HCoV-NL63.

The terms “sequence identity” or “identity” and “identical” before the background of proteins as used herein mean that two sequences are identical if they exhibit the same length and order of amino acids. The percentage of identity typically describes the extent, to which two sequences are identical, i.e. it typically describes the percentage of amino acids that correspond in their sequence position to identical amino acids of a reference sequence. For the determination of the degree of identity, the sequences to be compared are considered to exhibit the same length, i.e. the length of the longest sequence of the sequences to be compared. This means that a first sequence consisting of 8 amino acids is 80% identical to a second sequence consisting of 10 amino acids comprising the complete first sequence.

The term “aptamer” as used herein refers to artificial proteins selected or engineered to bind specific target molecules, in the present case to a spike protein, wherein the spike protein is characterized in that (i) it is from a coronavirus and (ii) it binds to ACE2” (e.g. the aptamer binds to the spike protein from SARS-CoV-2 and/or from SARS-CoV-1). Such proteins comprise one or more peptide loops of variable sequence displayed by a protein scaffold. They are typically isolated from combinatorial libraries and often subsequently improved by directed mutation or rounds of variable region mutagenesis and selection.

The term “vector” as used herein refers to a circular or linear, single-stranded or double-stranded nucleic acid, in particular DNA or RNA. Such a vector typically comprises further genetic information encoding further proteins, such as e.g. viral proteins that are necessary for the transduction of a host cell. If a simple DNA vector is referred to, this is typically a plasmid that comprises in particular certain markers for selection and/or detection, and optionally an origin of replication. If the vector is an mRNA, such mRNA typically comprises the typical elements of an mRNA that are required for a complete translation of the mRNA into the encoded T cell receptor fusion construct, such as e.g. a 5′ CAP structure, a 5′ and 3′ UTR, and a polyA-tail. The mRNA may comprise modified nucleotides that are commonly used in order to stabilize the mRNA.

### 2. Further Embodiments

1. A T cell receptor fusion construct comprising
   (a) a peptidic moiety binding to the spike protein from SARS-CoV-2 and/or SARS-CoV-1; and
   (b) a peptidic moiety selected from the group consisting of T cell receptor α chain, T cell receptor β chain, CD3γ, CD3ε, CD3δ, and variants of any of the foregoing.
2. The T cell receptor fusion construct according to embodiment 1, wherein the construct further comprises a peptidic linker between the peptidic moiety (a) and the peptidic moiety (b).
3. The T cell receptor fusion construct according to embodiment 1 or 2, wherein the construct further comprises a signal peptide before the peptidic moiety (a).
4. The T cell receptor fusion construct according to any one of embodiments 1 to 3, wherein the peptidic moiety (b) comprises an extracellular region, a transmembrane region and an intracellular region.
5. The T cell receptor fusion construct according to any one of embodiments 1 to 4, wherein the peptidic moiety (a) is selected from the group consisting of an antibody-derived fragment, an aptamer, and a receptor or a binding domain thereof.
6. The T cell receptor fusion construct according to embodiment 5, wherein the peptidic moiety (a) is a receptor or a binding domain thereof and wherein the receptor is ACE2.
7. The T cell receptor fusion construct according to any one of embodiments 1 to 6, wherein the peptidic moiety (a) comprises the amino acid sequence of SEQ ID NO: 2 or a sequence that is at least 85% identical thereto.
8. The T cell receptor fusion construct according to embodiment 5, wherein the peptidic moiety (a) is an antibody-derived fragment and wherein the antibody-derived fragment is a single chain fragment (scFv).
9. The T cell receptor fusion construct according to embodiment 8, wherein the single chain fragment comprises the amino acid sequence of SEQ ID NO: 5 and the amino acid sequence of SEQ ID NO: 6.
10. The T cell receptor fusion construct according to embodiment 9, wherein the single chain fragment (scFv) further comprises a peptidic linker between the amino acid sequences of SEQ ID NO: 5 and SEQ ID NO: 6.
11. A vector comprising the genetic information encoding a T cell receptor fusion construct according to any one of embodiments 1 to 10.
12. The vector according to embodiment 11, wherein the vector is selected from the group consisting of a lentiviral vector, a DNA vector, an RNA vector, a plasmid vector, a cosmid vector, a herpes virus vector, a measles virus vector, an adenoviral vector, and a retrovirus.
13. A process of transfecting or transducing T cells with a vector according to embodiment 11 or 12, wherein the T cells are collected and cultivated ex vivo, thereafter transfected or transduced with a vector according to embodiment 11 or 12, and thereafter grown and expanded ex vivo.
14. A modified T cell comprising a T cell receptor fusion construct according to any one of embodiments 1 to 10.
15. The T cell receptor fusion construct according to any one of embodiments 1 to 10, or the vector according to embodiment 11 or 12, or the modified T cell according to embodiment 14 for use in the treatment of a disease, preferably for use in the treatment of coronavirus disease 2019 (COVID-19) or the severe acute respiratory syndrome (SARS) disease.

### 3. EXAMPLES

The following Examples are merely illustrative and shall describe the present invention in a further way. These Examples shall not be construed to limit the present invention thereto.

### Example 1: Engineering of SARS-CoV-2-Specific TRuCs

Approach 1: Since the S protein binds to human ACE2, the part of ACE2 that binds to the S-protein (Van et al., 2020), including the signal peptide, was fused to human CD3ε. The complete extracellular part of ACE2 was not utilized, because this part also includes an ACE2-ACE2 dimerization motif and inclusion of a dimerization motif into the εTRuC might lead to aberrant signalling by the εTRuC-containing TCR. Hence, amino acids 1-615 of human ACE2, including the signal peptide, were used and fused to human CD3c using two different linker lengths (FIG. 3A) followed by a T2A peptide and GFP. The expected affinity of the protein S-ACE2 interaction is 15 nM (Tai et al., 2020). The cDNA sequence of the new constructs was integrated into a lentiviral vector with the EF1α promoter. The constructs are called ACE2I-εTRuC (I denotes long linker) and ACE2s-εTRuC (s denotes short linker).

Approach 2: An antibody called CR3022 was identified that binds to the S protein from SARS-CoV-1 with an affinity of <0.1 nM (Yuan et al., 2020) and to the S protein from SARS-CoV-2 with an affinity of 6 nM (Wrapp et al., 2020). Amino acids 1-133 from the light chain, including the signal peptide, were fused to a linker and to amino acids 20-136 from the heavy chain, excluding the signal peptide, and to CD3c followed by a T2A peptide and GFP (FIG. 3B). The CR3022 sequences were taken from a publication (ter Meulen et al., 2006). Again, the cDNA sequence of the new construct was part of a lentiviral vector with the EF1α promoter. The construct is called herein either αSLH-εTRuC (a denotes anti, S denotes the S protein and LH the VL and VH regions) or αS-εTRuC (without explicitly mentioning the “LH”). The terms “αSLH-εTRuC” and “α5-εTRuC” are therefore used interchangeably herein and refer to the same construct.

### Example 2: Expression of the SARS-CoV-2-Specific εTRuCs on T Cells

Jurkat is a human T cell line that expresses a TCR on its surface (Abraham and Weiss, 2004). When the TCR's subunit CD3c is missing a complete TCR cannot assemble and a TCR is not ex pressed on the cell surface. Here, Jurkat cells were used that lack CD3c due to the CRISPR technology (Jurkat CD3εKO) and that consequently do not express a TCR (FIG. 4). These cells were transduced with lentiviruses encoding for the TRuCs discussed in example 1. As a control we also used a lentivirus that only encodes for GFP (mock) or for the αCD19-εTRuC (data not shown). Cells were sorted for GFP-positive cells, since these were the transduced ones. Staining with an anti-CD3 antibody shows that a TCR was expressed in the εTRuC-transduced cells. This indicates that the εTRuCs integrated into a full TCR that was then exported to the cell surface. These TCRs are the ones that contain the S protein binding domains. Co-purification of the endogenous TCR subunits TCRβ and CD3ζ with the εTRuCs demonstrated that the εTRuCs assemble into a TCR and the corresponding Western blot confirmed the expected sizes of the εTRuCs (data not shown). Indeed, when these Jurkat cells were stimulated with target cells expressing the S protein from SARS-VoV-2, the Jurkat cells were activated as measured by CD69 upregulation (FIG. 5, up per panels). If the target cells did not contain the S protein, the εTRuC-transduced Jurkat cells were not activated (FIG. 5, lower panels). In conclusion, the SARS-CoV-2- and SARS-CoV-1-specific εTRuCs were expressed and integrated into a complete TCR. They were stimulated by target cells containing the SARS-CoV-2 S protein.

### Example 3: Killing of Cells Displaying the SARS-CoV-2 S Protein by the New εTRuC-T Cells

To test for the cytotoxic capability of T cells expressing the SARS-CoV-2 and SARS-CoV-1-specific εTRuCs, expanded primary human T cells were transduced to express the new εTRuCs. The αSLHεTRuC was expressed well (data not shown), whereas expression of the ACE2-εTRuC was below detection limit (data not shown). The primary T cells were co-cultured with target cells expressing or not the S protein from SARS-CoV-2. After 6 hours the percentage of killed target cells was quantified (FIG. 6). The unspecific spontaneous killing was measured using the mock transduced T cells. T cells expressing the αSLH-εTRuC killed more target cells expressing the S protein than mock cells (upper panel, p=0.004). As a control, when the target cells did not express the S protein, no enhanced killing over the mock control was observed (FIG. 6, lower panel). Although the primary T cells expressed very low levels of the ACE2-εTRuCs, they were able to kill target cells that expressed the S protein better than the mock T cells did (FIG. 6). The effect was observed with both εTRuCs (ACE2I-εTRuC and ACE2s-εTRuC) and in five independent experiments using two different donors (data not shown). These data demonstrate that the re-programming of primary T cells to kill target cells that display the SARS-CoV-2 S protein was successful.

### Example 4: The New εTRuCs Re-Program Jurkat Cells to Recognize S-Expressing Cells

Human Ramos-null B cells (with a deletion of the B cell receptor (He et al., 2018)) expressing SARS-CoV-2 S on their surface were also used to test whether the S-specific εTRuCs binding would lead to T cell activation. These cells could activate our S-specific εTRuC-expressing Jurkat cells as seen by upregulation of the activation marker CD69 (FIG. 7B). Similarly, expression of the alpha (6.1.1.7) and beta (B.1.351) variants of the S protein of SARS-CoV-2, but not of the common cold human corona viruses HCoV-0C43 or -229E, led to activation of our εTRuC-expressing Jurkat cells (FIG. 7B). The S protein of SARS-CoV-1 or HCoV-NL63 weakly stimulated our T cells. This is in line with the ability of the different S proteins to bind to ACE2 and the antibody CR3022 (Delmas et al., 1992; Hofmann et al., 2005; Tian X et al., 2020; Yeager et al., 1992). As a control, Ramos cells without S did not activate these Jurkat cells. Since Ramos cells express CD19, the αCD19-εTRuC Jurkat cells were activated by all Ramos cell lines (FIG. 7B). In conclusion, Jurkat cells expressing the SARS-CoV-2-specific εTRuCs recognize and are activated by target cells expressing S from SARS-CoV-2.

### Example 5: The Novel Primary εTRuC T Cells Lyse S-Containing Target Cells

Next, expanded primary human T cells were transduced to express the εTRuCs, in order to test for the cytotoxic capability of these T cells. All εTRuCs were expressed well on the surface of the T cells (FIG. 8A), which were CDB+ and CD4+ T cells (data not shown).

As an example to look at target cell killing, we chose the αS-εTRuC T cells which were co-cultured with Ramos cells that express a chimeric SARS-CoV-2 S protein with a C-terminal (cytosolic) fusion to the red fluorescent protein mScarlet (S-mScarlet, FIG. 8B). Hence, transduced T cells (green) can be distinguished from the target cells (red) by confocal fluorescent imaging (FIG. 8C). After contact of the αS-εTRuC T cells with the S-expressing Ramos cells, a stable cell conjugate was formed that lasted on average 153 min (FIG. 8D). Most likely an immunological synapse was formed as seen by the polarisation of the lysosomes (stained in magenta) towards the target cells (FIG. 8C, upper row, second image). In most cases, the S-expressing Ramos cells were killed as detected by membrane blebbing (upper row, last image) that is typical for apoptosis. When the target cell was killed, this took on average 121 min (FIG. 8E). As a control, both mock transduced T cells and untransduced T cells, had only short contact to the Ramos cells (average 10 min) and left without cytotoxic activity (FIG. 8C and D).

All S-specific εTRuC and control engineered primary T cells were co-cultured with the Ramos cells, which served as target cells, expressing or not the SARS-CoV-2 S, to quantify the cytotoxic activity. The target cells also expressed the firefly luciferase and BFP (data not shown). If they were lysed, luciferase activity was lost, serving as a readout for the killing of the target cells by the T cells. Indeed, all εTRuC T cells lysed the S-expressing Ramos cells very efficiently, whereas mock T cells did not (FIG. 8F). As expected only the αCD19-εTRuC T cells killed the Ramos cells that did not express the S protein. Ramos cells expressing S of the alpha (B.1.1.7) and beta (B.1.351) variants were also lysed very efficiently, but not those expressing S from HCoV-NL63, -OC43 or -229E (FIG. 8F). There was a weak, but clear, activity also against Ramos cells expressing S from SARS-CoV-1. As above, we did not observe any differences between the ACE2I-, ACE2s- and αS-εTRuCs. Together these data demonstrate that we were able to redirect primary T cells to kill efficiently target cells that display SARS-CoV-2 S.

### Example 6: The S-Specific Primary εTRuC T Cells are Activated by S-Expressing Target Cells

Incubation of our engineered primary T cells with the Ramos cells expressing S from different coronaviruses showed that the SARS-CoV-2 S protein led to upregulation of CD69 on the T cells (FIG. 9A). The same was observed for S of the alpha (B.1.1.7) and beta (B.1.351) variants. The ones from SARS-CoV-1 and HCoV-NL63 caused a slight CD69 upregulation (it should be noted in this respect that the S protein from both, SARS-CoV-1 and HCoV-NL63, binds to ACE2), whereas the ones from HCoV-OC43 or -229E were inactive (it should be noted in this respect that the S protein from both, HCoV-OC43 and -229E, does not bind to ACE2). In the T cells that are not specific for the SARS-CoV-2 S, CD69 was not upregulated by any S-expressing Ramos cell (FIG. 9A). We also show that the engineered T cells that were activated by SARS-CoV-2 S-expressing target cells secreted the cytokines IL2, IFNγ, IFNα and TNFα (FIG. 9B). As a control, the mock T cells did not produce any of those cytokines nor did a co-culture with Ramos cells that do not express any S (data not shown). In conclusion, our re-programmed T cells with the new S-specific εTRuCs could be activated by S-expressing target cells.

### Example 7: The New S-Specific Primary εTRuC T Cells Kill SARS-CoV-2-Infected Cells

Having shown that the S-specific εTRuC T cells kill S-expressing Ramos cells, we wanted to test whether these TRuC T cells are also able to kill SARS-CoV-2-infected Vero E6 cells (FIG. 10A, up per panel). To quantify killing as above, Vero E6 cells were lentivirally transduced to express luciferase and BFP bicistronically separated by an IRES sequence (called VeroLucBFP cells, data not shown) and were infected with the authentic B.1 SARS-CoV-2 using a multiplicity of infection (MOI) of 0.05. After one hour, remaining virus was washed away, T cells were added and the luciferase signal was recorded (FIG. 10A, lower panel). When the T cells had been transduced with the S-specific εTRuCs (FIG. 10B, green and blue) the luciferase activity of the infected VeroLucBFP culture was reduced at 2 and 4 hours compared to the one treated with the mock and αCD19-εTRuC T cells (red and orange). The reduction of luciferase activity at early time points indicated that S-specific εTRuC T cells killed infected VeroLucBFP cells specifically, since treatment with the S-specific εTruC T cells did not cause a decrease in luciferase activity in uninfected VeroLucBFP cells beyond background (FIG. 10C). Elimination of infected cells within 2-4 hours is in line with the fast killing of Ramos cells expressing the S (FIG. 8E).

Similar results were obtained when the human colorectal adenocarcinoma cell line Caco-2 or the human lung epithelial cell line Calu-3 cells were transduced with luciferase and taken as host cells for the virus infection (data not shown). In conclusion, engineered primary human T cells expressing ACE2s-, ACE2I- and αS-εTRuCs were successfully re-programmed to recognize and kill cells infected with SARS-CoV-2.

### Example 8: S-Specific εTruC T Cells Prevent the Loss of Luciferase Activity in Infected Cells

Next, we repeated the treatment of infected VeroLucBFP cells with our T cells, but recording luciferase activity for 72 hours. With the control T cells (mock and αCD19-εTRuC) the luciferase signal started to decrease 6 hours post infection (FIG. 10D, red and orange). A similar drop in luciferase activity was observed when no T cells were added (black line), showing that T cells per se did not recognize SARS-CoV-2-infected cells. At 72 hours post infection no luciferase activity was detected anymore. This loss of luciferase signal was prevented by the presence of the S-specific εTRuC T cells (green and blue). In fact, at 48 and 72 hours post infection the infected and treated cultures had similar luciferase activity as the uninfected ones (FIG. 10D and E), demonstrating that the TRuC treatment resulted in a sustained luciferase expression.

Similar results were obtained when a different ratio of T cell to VeroLucBFP cells was used (data not shown) or when Caco-2 or Calu-3 cells were infected (data not shown). In all cases, our treatment completely rescued the infected cell cultures in terms of the virus-induced loss of luciferase activity.

### Example 9: SARS-CoV-2 Causes a Translation Shutdown in VeroLucBFP Cells

In theory, the loss of luciferase signal may result from three different mechanisms: (i) By killing of VeroLucBFP cells by the S-specific TRuC T cells (as above); (ii) By reducing the VeroLucBFP cell number by either SARS-CoV-2-induced cell death (Park et al., 2020) or reduced cellular proliferation rates (our graphs depict the luciferase activity of the wells in relation to the wells with the un infected cells); (iii) By the SARS-CoV-2-mediated suppression of host protein synthesis (Hsu et al., 2021; Lapointe et al., 2021; Puray-Chavez et al., 2020; Thorns et al., 2020; Yuan et al., 2020b), called translation shutdown. This mechanism acts at the single cell level.

To monitor the effect of translation shutdown, we blocked protein synthesis in VeroLucBFP cells with puromycin, which causes premature chain termination during mRNA translation (Pestka, 1971). Indeed, puromycin-treatment suppressed luciferase activity before the occurrence of cell death (FIG. 11A). To directly examine whether a strong translation shutdown in infected cells occurred, we determined the BFP expression in SARS-CoV-2-infeceted VeroLucBFP cells. BFP allows us to investigate the translation shutdown at the single cell level by flow cytometry. Indeed, at 72 hours post infection when no luciferase activity was detectable (FIG. 10D), the BFP expression was similarly lost (in the control cultures that were treated with mock or αCD19-εTRuCs T cells) (FIG. 11B). This suggests that a complete translation shutdown is induced by SARS-CoV-2 in the VeroLucBFP cells, as seen earlier in other cells (Hsu et al., 2021; Lapointe et al., 2021; Puray-Chavez et al., 2020; Thorns et al., 2020; Yuan et al., 2020b). This loss of BFP-expression was not observed in uninfected VeroLucBFP cells (FIG. 11B). SARS-CoV-2 infection also led to slightly reduced VeroLucBFP cell numbers at 72 hours (data not shown), most likely due to viral lysis of the host cells (Park et al., 2020).

### Example 10—S-Specific εTRuC T Cells Prevent the Translation Shutdown, but not Viral Replication

The loss of luciferase activity at later time points post infection, which is indicative for the translation shutdown, was drastically reduced upon S-specific εTRuC T cell treatment (FIG. 11D). Indeed, treatment of infected VeroLucBFP cells with ACE2s- or αS-εTRuC T cells, also prevented the loss of BFP expression (FIG. 11B). The infected and treated VeroLucBFP cells had similar BFP values as the uninfected ones. Assuming that the S-specific εTRuC-treatment would prevent the translation shutdown by an killing of all infected VeroLucBFP cells immediately post infection, we expected no viral replication and spread in the tissue culture. Surprisingly, although some killing of infected cells was detected at early time points (FIG. 11B), SARS-CoV-2 replicated equally well independent of our treatment. This was seen by determining the virus titer of the culture supernatants at different time points post infection (FIG. 11C) and by SARS-CoV-2 nucleocapsid protein (N) staining in infected cells (data not shown). In conclusion, at later time points post infection (8 to 72 hours) the virus replicates, infects new VeroLucBFP cells and leads to a suppression of the host protein synthesis in the VeroLucBFP cell culture. Importantly, this shutdown was completely prevented by treatment with our engineered T cells.

Lastly, we sought to get insight into how the S-specific εTRuC T cell blocks the virus-induced translation shutdown. To this end, we activated ACE2s- or αS-εTRuC T cells by co-culturing them with S-expressing Ramos cells for 24 hours. We then collected the medium that should contain factors that these T cells secrete and treated uninfected and SARS-CoV-2-infected VeroLucBFP cells with this “conditioned” medium. Upon treatment of SARS-CoV-2-infected cells with the ACE2s- or αS-εTRuC T cell “conditioned” medium, the loss of luciferase activity and of BFP expression was reduced compared to cells incubated with “conditioned” medium derived from mock T cells (FIGS. 12A and 12B). These data suggest that our engineered T cells prevent virus-induced protein shutdown in VeroLucBFP cells by secreting a protective factor or a cocktail of different factors instead of killing infected cells. This is in line with the observation that the T cells also prevented the translation shutdown, when using a low MOI, where the killing of infected cells at the early time point was not visible (data not shown).

### Example 11: Materials and Methods Used in the Above Examples

Generation of CAR Constructs/Molecular Cloning of the εTRuC Constructs.

The plasmid pcDNA3 ACE2 was a gift from Michael Reth. The sequences encoding for the CR3022 heavy and light chains were synthesized as gBlocks (IDT) according to the published se quences (ter Meulen et al., 2006).

The lentiviral vectors coding for the different εTRuCs were generated by exchanging the sequence encoding anti-hCD19 scFv (FMC63) fused to human CD3c (amino acids 23-207) in the plasmid p526 anti-CD19.2) (Baeuerle et al., 2019), with sequences encoding either human ACE2 (amino acids 1-615) or the CR3022 scFv (light chain amino acids 18-133, heavy chain amino acids 20-136).

In detail, pOSY120, encoding for the ACE2I-ETRuC, was generated by Gibson assembly of the Xhol-Xbal fragment of p526 anti-αCD19 (Baeuerle et al., 2019) and the PCR fragment using the primers of SEQ ID NOs:26 and 27 on the human ACE2 sequence as a template. pOSY121, encoding for the ACE2s-ETRuC, was generated by Gibson assembly of the Xhol-Xbal fragment of above and the PCR fragment using the primers of SEQ ID NOs: 26 and 28 on the human ACE2 sequence. pOSY123, encoding the αS-εTRuC, was generated by Gibson assembly of the Xhol-Xbal fragment of above and, the PCR fragment using the primers of SEQ ID NOs: 29 and 30 from a gBlock of CR3022 VL (Integrated DNA Technologies) and the PCR fragment using the primers of SEQ ID NOs: 31 and 32 from a gBlock of CR3022 VH (Integrated DNA Technologies). All plasmid sequences were verified by Sanger sequencing (Eurofins Genomics).

Molecular Cloning of the Luciferase-BFP and S Protein-mScarlet Vectors.

For the molecular cloning of the pHRSIN-CS-Luc-IRES-mTagBFP2 vector, the previously de scribed pHRSIN-CS-Luc-IRES-emGFP vector (a kind gift from A. Rodriguez, Universidad Autonoma Madrid, Spain) was digested with BstX1 and Not1 restriction enzymes to remove the emGFP. mTagBFP2 was amplified from the previously described pHRSIN-CS-IRES-mTagBFP2 plasmid (Dang et al., 2020) and BstX1- and Not1-specific overhangs were added using PCR. The final expression vector was generated by Gibson assembly using the pHRSIN-CS-Luc-IRES as recipient vector and the amplified mTagBFP2 as insert. The integrity of the plasmid was verified by Sanger sequencing. The retroviral expression vector encoding the S protein of SARS-CoV-2 fused at is cytoplasmic tail to mScarlet was cloned as follows. The cDNA of the S protein was taken from the plasmid pCG1-CoV-2019-S with a codon-optimized sequence (Lapuente et al., 2021) and cloned into the pMIG vector by Gibson assembly and the cDNA of mScarlet was ligated into the vector. All plasmid sequences were verified by Sanger sequencing (Eurofins Genomics).

Generation of Lentiviruses.

10⁷ HEK293T cells were plated on a 15 cm plate in 20 ml of DMEM, distributed evenly and incubated at 37° C. and 7.5% CO₂. After 24 h, the medium was changed and HEK293T cells were transfected with the indicated CoV-2-specific TRuC lentiviral constructs and the packaging plasmids pMD2.G (envelope) and pCMVR8.74 (gag/pol) using PEI (Polysciences) transfection. The virus-containing supernatant was collected 24 and 48 h after transfection and was concentrated by a 10% sucrose gradient (supplemented with 0.5 mM EDTA) centrifugation for 4 h at 10,000 r.p.m. and 8° C. After centrifugation, supernatant was discarded and the virus pellet was resuspended in 100 μl of RPMI medium and stored at −80° C.

Cells

Jurkat (human T cell line), Ramos (human Burkitt's lymphoma line), VeroE6 (kidney epithelial cell line form the African green monkey), CaCo-2 (human colorectal adenocarcinoma line) and CaLu3 (human lung epithelial cell line) cells and their derivatives were grown with complete RPMI 1640 medium and 10% fetal calf serum (FCS) at 37° C. and 5% CO₂. Jurkat CD3ε knock out (KO) cells were generated by standard CRISR/Cas9 technology and the generation of the Ramos cells ex pressing the different S proteins will be described elsewhere. Jurkat CD3ε KO, Ramos, Vero E6, CaCo-2 and CaLu-3 cell lines were transduced with a multiplicity of infection (MOI) of 5 with the lentiviruses indicted and sorted by flow cytometry when necessary.

To obtain the expanded human T cells, peripheral blood mononuclear cells (PBMCs) were isolated from blood of a healthy donors by density-gradient centrifugation and grown in RPMI 1640 medium supplemented with 10% FCS and 1000 U/ml recombinant IL-2 (PeproTech) and activated with 1 μg/ml anti-CD3 and anti-CD28 antibodies. At 48-72 h the remaining PBMCs were mostly T cells (>99%) and lentivirally transduced by spin infection with 5 μg/ml of protamine sulfate with a MOI of 4. Transduced T cells were tested using flow cytometry by staining with anti-Fab for the αCD19-ETRuC, anti-ACE2 for the ACE2s- and ACE2I-εTRuCs, and anti-human IgG for the αS-εTRuC. Cells were expanded in complete RPMI 1640 medium with 10% FCS and were given 100 U/ml IL-2 every third day. Cells were used until day 17 post transduction.

To generate S-mScarlet expressing Ramos cells, the retroviral vector pMIG encoding S-mScarlet and a vector encoding the ecotropic packaging protein were co-transfected (500 ng each) into Plat-E cells with the PolyJet transfection reagent (Signagen). After two days, the supernatant was collected, filtered and mixed 1:1 with 300.000 Ramos-null cells that express the ecotropic recep tor. Cells were sorted for mScarlet expression.

The transduction of Ramos cells with LucBFP was done as follows. Retroviral transduction of the human Ramos-null B cells (with a deletion of the B cell receptor (He et al., 2018)) with vectors en coding for the different S proteins will be published elsewhere. These Ramos-null cells expressing S proteins or not were lentivirally transduced with pHRSIN-CS-Luc-IRES-mTagBFP2 as briefly de scribed above.

Flow Cytometry

The following antibodies were used for flow cytometry staining in a 96-well format: PE-labelled anti-human CD4 (Beckman Coulter, #A07752), APC-labelled anti-human CD8 (Beckman Coulter, #IM2469), APC- or PacificBlue-labelled anti-human CD3 (BioLegend, #300434), PE-labelled anti-human CD69 (Life Technologies, #MHCD6904), anti-human ACE2 (R&D Systems, #HK0320042), PE-labelled anti-human IgG (Southern Biotech, #2040-09) and biotin-labelled anti-Fab (Invitrogen, #31803). APC-coupled Streptavidin (Biolegend, #105213) and APC-labelled donkey anti-goat IgG (Southern Biotech, #6420-05) served as a secondary reagent. Cells were measured on the flow cytometer Attune N×T and the data were analyzed by FlowJo.

For flow cytometry of virus-infected cells post-treatment, the cells were fixed with methanol and paraformaldehyde and then washed 4 times before measuring on AttuneNxT.

Co-Immunopurification and Western Blotting

Following antibodies were used for biochemical analysis: anti-TCRα (clone H-1, Santa Cruz, #sc515719), anti-TCRβ (clone H-197, Santa Cruz, #sc-9101), anti-CD3γ (clone EPR4517, Epitomics, #3256-1), anti-CD36 (clone F-1, Santa Cruz, #sc-137137,), anti-CD3E (clone M20, Santa Cruz, #sc1127), anti-CD3ζ (serum 449), horseradish peroxidase (HRPO)-coupled anti-mouse IgG (Thermo Fisher, #32430), HRPO-coupled anti-goat IgG (Thermo Fisher, #31402), and HRPO-coupled anti-rabbit IgG (Thermo Fisher, #31460). Protein G-coupled sepharose (#17-0618-01) and Protein A-coupled sepharose (#17-5138-01) beads were from GE Healthcare and the protease inhibitor cocktail was from Sigma.

3×10⁷ cells were lysed in 0.4 ml lysis buffer containing 20 mM Tris-HCl pH8, 137 mM NaCl, 2 mM EDTA, 10% glycerol, 1× protease inhibitor cocktail, 1 mM PMSF, 5 mM iodoacetamide, 0.5 mM sodium orthovanadate, 1 mM NaF, and 0.5% Brij96 for 30 min at 4° C. followed by 15 min centrifugation to pellet the nuclei and insoluble material. For the anti-CD3E immunoprecipitation 370 μl cleared cell lysate was incubated with 5 μl 50% protein A and protein G sepharose slurry (1:1) and 2 μg anti-CD3ε UCHT1 for 4 h at 4° C. After three washes, the immunoprecipitated material was separated by 12% reducing SDS-PAGE. The separated proteins were transferred to PVDF membranes by semi-dry transfer. After blocking with 5% milk in PBS containing 0.1% Tween-20 the membranes were incubated with antibodies against TCRα (1:1000), TCRβ (1:100), CD3γ (1:1000), CD36 (1:100), CD3ε (1:1000), CD3ζ (1:1000) in PBS-T followed by incubation with HRPO-conjugated secondary antibodies (1:10000). Western blot signals were recorded using an Image Quant LAS 4000 Mini from GE Healthcare Life Sciences, Boston, MA.

Activation Assays

Ramos cells expressing or not the different S proteins were co-cultured with the different Jurkat transductant cells at a 1:3 target-to-effector ratio for 9 h. Cells were stained with anti-CD69 anti bodies and measured by flow cytometry. The BFP-positive Ramos cells were gated out to ensure that only the T cells are analysed.

To quantify cytokines by ELISA, εTRuC-expressing primary T cells and S-expressing Ramos cells were co-cultured for 24 h. The following cytokines were measured according to the instructions of each ELISA kit: TNFα (Invitrogen, #88-7346-88), IFNγ (Invitrogen, #88-7316-88), IFNα (Invitrogen, #BMS216) and IL2 (Invitrogen, #88-7025-88).

Transduction and Sorting of Jurkat CD3εKO Cells.

105 Jurkat CD3εKO cells were seeded in a 24-well plate and lentivirally transduced using spin infection in the presence of 5 μg ml-1 protamine sulfate (Sigma) and 30 μl of the concentrated virus. The cells were allowed to grow for 72 hours before testing for GFP and surface expression of the TCR by flow cytometry. 5 days post lentiviral transduction of the Jurkat CD3εKO cells, the cells were stained with an APC-labelled anti-CD3 antibody (clone UCHT1) and taken for sorting of GFP and CD3 double positive cells.

Upregulation of the Activation Marker CD69.

104 of the Jurkat CD3εKO transductants were seeded in a 96-well plate (Greiner Bio) with 2×104 of either Ramos cells expressing the SARS-CoV-2 S protein (+S) or not (−S) and incubated at 37° C., 5% CO₂ for 9 hours. Subsequently, the cells were stained using PE-labelled anti-hCD69 and APC-labelled anti-hCD3 antibodies. Fluorescence was quantified using flow cytometry and the data were analysed by FlowJo.

Primary Human T Cell Activation, Transduction and Expansion.

Peripheral blood mononuclear cells (PBMCs) were purified from fresh blood of healthy donors using density centrifugation (Ficoll-Paque). PBMCs were counted, resuspended in medium supplemented with 1,000 U ml-1 recombinant human IL-2 (PeproTech) and activated with antiCD3/CD28 (1 μg ml-1). At 48-72 h after activation, the remaining PBMCs were mostly T cells (>99%). The expanded primary T cells were then lentivirally transduced using spin infection in the presence of 5 μg ml-1 protamine sulfate (Sigma), 1,000 U ml-1 IL-2 with a multiplicity of infection of 4 (unless otherwise indicated). Transduced T cells were tested for TRuC expression 5-7 days after transduction using biotinylated primary goat anti-mouse F(ab′)2 (Invitrogen) followed by streptavidin-APC (BioLegend). Cells were cultured in medium supplemented with 100 U ml-1 IL-2 for a maximum of 7 days after transduction before use for the killing experiments.

Cytotoxicity Assay.

For the Bioluminescence-based killing assay, luciferase-expressing Ramos cells that either also expressed the SARS-CoV-2 spike protein or not, were plated at a concentration of 10⁴ cells ml-1 in 96-well flat bottom plates in triplicates. Then, 75 μg m1-1 D-firefly luciferin potassium salt (Bio-synth) was added to the tumor cells and bioluminescence (BLI) was measured in the luminometer (Tecan infinity M200 Pro) to establish the BLI baseline. Right after, TRuC-expressing T cells were added at an effector-to-target ratio of 5:1 and incubated for 8 or 24 h (as indicated) at 37° C. BLI was measured as relative light units (RLUs). RLU signals from cells treated with 1% Triton X-100 indicate maximal cell death. RLU signals from tumor cells without TRuC T cells determine spontaneous cell death. Percent specific lysis (specific killing) was calculated with the following formula: percentage specific lysis=100×(average spontaneous death RLU−test RLU)/(average spontaneous death RLU−average maximal death RLU).

Time-Lapse Microscopy

Ramos B cells were retrovirally transduced with a construct encoding spike protein tagged with the fluorophore mScarlet at the C-terminus and sorted by FACS (Bio-Rad S3e Cell Sorter). For the experiment, mScarlet-spike-expressing Ramos B cells were washed 3 times in PBS. 15,000 cells in PBS were seeded into both wells of a 2 well-culture insert placed on a 35 mm dish (Ibidi). PBS facilitated the attachment of Ramos B cells onto the ibi-treated dish surface. T cells were incubated with 50 nM Lysotracker Deep Red (Invitrogen) for 15 min at a 37° C. incubator with 5% CO₂, washed twice and resuspended in phenol red-free RPMI containing 2 mM GlutaMAX (Gibco), 10% FCS (PAN), 50 U/ml Pen-Strep (Gibco) and 10 mM HEPES (Gibco). 7,500 mock- and anti-spike-transduced T cells were seeded into the 2 well-culture insert with Ramos B cells after re moving the PBS. Mock- and anti-spike-transduced T and Ramos B cell interactions were recorded in parallel using a Zeiss Observer microscope equipped with a 40× oil objective and Zen Blue software. Single plane images in 4 channels (GFP, mScarlet, Lysotracker deep red, brightfield) were acquired every 2 or 3 min for 4 h within an incubator set to 37° C. and 5% CO₂. Multi-position videos were converted to TIFF and transferred to Images. Duration of interaction between T and B cells and the time it takes for an anti-spike-transduced T cell to kill mScarlet-spike-expressing Ramos B cell were quantified manually. Cell death was assessed by apoptotic morphology and the subsequent halt in cellular movement from the brightfield images.

SARS-CoV-2 Infection and Measurement of Luciferase Activity

A bioluminescence based cytotoxicity assay was performed with transduced primary cells co-cultured with VeroE6 (kidney epithelial cells extracted from an African green monkey), CaCo-2 (human colorectal adenocarcinoma cells) and CaLu-3 (human lung cancer cell line) cell lines. Each of these cell lines were infected with the B.1 SARS-CoV-2 virus at the indicated MOI. A control setup had the same cell lines without the infection. 104/pi of target (Vero E6, CaCo-2 and CaLu-3) cells were plated in a 96-well flat bottom plate (Corning, #3917). 75 μg/ml D-firefly luciferin potassium salt (Biosynth) was added to it and bioluminescence (BLI) was measured in the luminometer (Tecan infinity M200 Pro) to establish the BLI baseline. Right after, TRuC-expressing T cells (effector cells) were added at varying effector-to-target ratio (as indicated) and incubated until 72 h post infection (as indicated) at 37° C. BLI was measured as relative light units (RLUs). RLU signals from cells treated with 1% Triton X-100 indicate maximal cell death. RLU signals from tumor cells without TRuC T cells determine spontaneous cell death. Percent specific lysis (specific cytotoxicity) was calculated with the following formula:

percentage specific lysis=100−(100×(average spontaneous deathRLU−testRLU)/(average spontaneous deathRLU−average maximal deathRLU)).*Here,spontaneous death was considered of only the target cells without the virus and withoutT-cells.

Immunofluorescence and Viral Titers

Virus containing supernatant was harvested at the indicated time points and the viral titer was determined on VeroLucBFP cells by indirect-immunofluorescence. Briefly, VeroLucBFP cells were seeded in 96-well plates at a density of 0.04×106 cells/well 24 h prior to infection. Infectious cell supernatants were diluted in tenfold dilution series in PBS containing 2% BSA in a 100 μl volume and subsequently incubated on VeroLucBFP. At 20 h post infection, the infectious supernatant was removed and cells were fixed using 4% formaldehyde for 30 minutes. Virus-infected cells were subsequently detected using anti-SARS-CoV nucleocapsid (N) rabbit antiserum (Rockland Immunochemicals, #200-401-A50) and secondary anti-rabbit IgG-coupled to Cyanine Cy3 (Jackson ImmunoResearch). Nuclei were stained with DAPI. Viral endpoint titers were evaluated by fluorescence microscopy.

To monitor SARS-CoV-2 spread upon εTRuC T cell-treatment, infected VeroLucBFP cells were fixed at the indicated time points post infection with 4% formaldehyde for 30 minutes and subjected to indirect-immunofluorescence as described. Fluorescence images were acquired using a Zeiss Observer.Z1 inverted epifluorescence microscope (Carl Zeiss) equipped with an AxioCamMR3 camera using a ×20 objective.

Statistics

Statistical significance of one sample in comparison to another (a control) was determined using a paired Student's t-test. All p values indicated with stars (*<0.05, **<0.005, ***<0.0005) were calculated using Prism v.6 software (GraphPad). Error bars show standard deviation in all graphs.

### REFERENCES

Abraham, R. T., and Weiss, A. (2004). Jurkat T cells and development of the T-cell receptor signal ling paradigm. Nat Rev Immunol 4, 301-308.
Baeuerle, P. A., Ding, J., Patel, E., Thorausch, N., Horton, H., Gierut, J., Scarfo, I., Choudhary, R., Kiner, O., Krishnamurthy, J., et al. (2019). Synthetic TRuC receptors engaging the complete T cell receptor for potent anti-tumor response. Nature communications 10, 2087.
Boyer, M. W. (2018). Chimeric antigen receptor T-cell therapy hits the market Immunotherapy 10, 911-912.
Dang, A. T., Strietz, J., Zenobi, A., Khameneh, HJ., Brandl, S. M., Lozza, L., Conradt, G., Kaufmann, S. H. E., Reith, W., Kwee, I., et al. (2020). NLRC5 promotes transcription of BTN3A1-3 genes and Vγ9Vδ2 T cell-mediated killing. iScience 24, 101900.
Delmas, B., Gelfi, J., L'Haridon, R., Vogel, L. K., Sjöström, H., Norén, O., and Laude, H. (1992). Aminopeptidase N is a major receptor for the entero-pathogenic coronavirus TGEV. Nature 357, 417-420.
Gudipati, V., Rydzek, J., Doel-Perez, I., Goncalves, V. D. R., Scharf, L., Königsberger, S., Lobner, E., Kunert, R., Einsele, H., Stockinger, H., et al. (2020). Inefficient CAR-proximal signaling blunts antigen sensitivity. Nat Immunol 21, 848-856.
Hardy, I. R., Schamel, W. W., Baeuerle, P.A., Getts, D. R., and Hofmeister, R. (2020). Implications of T cell receptor biology on the development of new T cell therapies for cancer. Immunotherapy 12, 89-103.

He, X., Klasener, K., lype, J. M., Becker, M., Maity, P. C., Cavallari, M., Nielsen, PJ., Yang, J., and Reth, M. (2018). Continuous signaling of CD79b and CD19 is required for the fitness of Burkitt lymphoma B cells. EMBO J 37, e97980.
Hofmann, H., Pyrc, K., van der Hoek, L., Geier, M., Berkhout, B., and Pöhlmann, S. (2005). Human coronavirus NL63 employs the severe acute respiratory syndrome coronavirus receptor for cellu lar entry. Proc Natl Acad Sci USA 102, 7988-7993.
Hsu, J. C., Laurent-Rolle, M., Pawlak, J. B., Wilen, C. B., and Cresswell, P. (2021). Translational shut down and evasion of the innate immune response by SARS-CoV-2 NSP14 protein. Proc Natl Acad Sci USA 118, e2101161118.
Hu, Y., Li, W., Gao, T., Cui, Y., Jin, Y., Li, P., Ma, Q., Liu, X., and Cao, C. (2017). The Severe Acute Respiratory Syndrome Coronavirus Nucleocapsid Inhibits Type I Interferon Production by Interfering with TRIM25-Mediated RIG-I Ubiquitination. J Virol 91, e02143-02116.
June, C. H., and Sadelain, M. (2018). Chimeric Antigen Receptor Therapy. The New England Journal of Medicine 379, 64-73.
Kamitani, W., Huang, C., Narayanan, K., Lokugamage, K. G., and Makino, S. (2009). A two-pronged strategy to suppress host protein synthesis by SARS coronavirus Nsp1 protein. Nat Struct Mol Biol 16, 1134-1140.Kupferschmidt, K., and Cohen, J. (2020). Race to find COVID-19 treatments acceler ates. Science 367, 1412-1413.
Lapointe, C. P., Grosely, R., Johnson, A. G., Wang, J., Fernandez, I. S., and Puglis, J. D. (2021). Dy namic competition between SARS-CoV-2 NSP1 and mRNA on the human ribosome inhibits translation initiation. Proc Natl Acad Sci USA 118, e2017715118.
Lapuente, D., Maier, C., Irrgang, P., Hubner, J., Peter, A. S., Hoffmann, M., Ensser, A., Ziegler, K., Winkler, T. H., Birkholz, T., et al. (2021). Rapid response flow cytometric assay for the detection of antibody responses to SARS-CoV-2. Eur J Clin Microbiol Infect Dis 40, 751-759.
Lei, X., Dong, X., Ma, R., Wang, W., Xiao, X., Tian, Z., Wang, C., Wang, Y., Li, L., Ren, L., et al. (2020). Activation and evasion of type I interferon responses by SARS-CoV-2. Nat Commun 11, 3810.
Narayanan, K., Huang, C., Lokugamage, K., Kamitani, W., Ikegami, T., Tseng, C. T., and S., M. (2008). Severe acute respiratory syndrome coronavirus nsp1 suppresses host gene expression, including that of type I interferon, in infected cells. J Virol 82, 4471-4479.
Park, W. B., Kwon, N.J., Choi, SJ., Kang, C. K., Choe, P. G., Kim, J. Y., Yun, J., Lee, G. W., Seong, M. W., Kim, N J., et al. (2020). Virus Isolation from the First Patient with SARS-CoV-2 in Korea. Korean Med Sci 35, e84.
Pestka, S. (1971). Inhibitors of ribosome functions. Annu Rev Microbiol 25, 487-562.
Puray-Chavez, M., Tenneti, K., Vuong, H. R., Lee, N., Liu, Y., Horani, A., Huang, T., Case, J. B., Yang, W., Diamond, M. S., et al. (2020). The translational landscape of SARS-CoV-2 and infected cells. bioRxiv 2020.11.03.367516
Sadelain, M. (2016). Chimeric antigen receptors: driving immunology towards synthetic biology. Curr Opin Immunol 41, 68-76.
Schamel, W. W., and Reth, M. (2012). Synthetic immune signaling. Curr Opin Biotechnol 23, 780-784.
Sparrer, K. M., and Gack, M. U. (2015). Intracellular detection of viral nucleic acids. Curr Opin Microbiol 26, 1-9.
Tai, W., He, L., Zhang, X., Pu, J., Voronin, D., Jiang, S., Zhou, Y., and Du, L. (2020). Characterization of the receptor-binding domain (RBD) of 2019 novel coronavirus: implication for development of RBD protein as a viral attachment inhibitor and vaccine. Cell Mol Immunol 17, 613-620.
Tanaka, T., Kamitani, W., DeDiego, M. L., Enjuanes, L., and Matsuura, Y. (2012). Severe acute respiratory syndrome coronavirus nsp1 facilitates efficient propagation in cells through a specific translational shutoff of host mRNA. J Virol 86, 11128-11137.
ter Meulen, J., van den Brin, E. N., Poo, L. L., Marissen, W. E., Leung, C. S., Cox, F., Cheung, C. Y., Bak ker, A. Q., Bogaards, J. A., van Deventer, E., et al. (2006). Human monoclonal antibody combination against SARS coronavirus: synergy and coverage of escape mutants. PLoS Med 3, e237.
Thoms, M., Buschauer, R., Ameismeier, M., Koepke, L., Denk, T., Hirschenberger, M., Kratzat, H., Hayn, M., Mackens-Kiani, T., Cheng, J., et al. (2020). Structural basis for translational shutdown and immune evasion by the Nsp1 protein of SARS-CoV-2. Science 369, 1249-1255.
Tian X, L., C., Huang, A., Xia, S., Lu, S., Shi, Z., Lu, L., Jiang, S., Yang, Z., Wu, Y., and Ying, T. (2020). Potent binding of 2019 novel coronavirus spike protein by a SARS coronavirus-specific human monoclonal antibody. Emerg Microbes Infect 9, 382-385.
Walls, A. C., Park, YJ., Tortorici, M. A., Wall, A., McGuire, A. T., and Veesler, D. (2020). Structure, Function, and Antigenicity of the SARS-CoV-2 Spike Glycoprotein. Cell 181, 281-292.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Xia H, C. Z., Xie, X., Zhang, X., Chen, J. Y., Wang, H., Menachery, V. D., Rajsbaum, R., and Shi, P. Y. (2020). Evasion of Type I Interferon by SARS-CoV-2. Cell Rep 33, 108234.
Yan, R., Zhang, Y., Li, Y., Xia, L., Guo, Y., and Zho, Q. (2020). Structural basis for the recognition of SARS-CoV-2 by full-length human ACE2. Science 367, 1444-1448.
Yousefi, O. S., Gunther, M., Horner, M., Chalupsky, J., Wess, M., Brandt, S. M., Smith, R. W., Fleck, C., Kunkel, T., Zurbriggen, M. D., et al. (2019). Optogenetic control shows that kinetic proofreading regulates the activity of the T cell receptor. eLife 8.
Yeager, C. L., Ashmun, R. A., Williams, R. K., Cardellichio, C. B., Shapiro, L. H., Look, A. T., and Holmes, K. V. (1992). Human aminopeptidase N is a receptor for human coronavirus 229E. Nature 357, 420-422.
Yuan, M., Wu, N.C., Zhu, X., Lee, C. D., So, R. T. Y., Lv, H., Mok, C. K. P., and Wilson, I. A. (2020). A highly conserved cryptic epitope in the receptor binding domains of SARS-CoV-2 and SARS-CoV. Science 368, 630-633.
Yuan, S., Peng, L., Park, JJ., Hu, Y., Devarkar, S. C., Dong, M. B., Shen, Q., Wu, S., Chen, S., Lomakin, I. B., et al. (2020b). Nonstructural Protein 1 of SARS-CoV-2 Is a Potent Pathogenicity Factor Redirecting Host Protein Synthesis Machinery toward Viral RNA. Molec Cell 80, 1055-1066.
Yuen, C. K., Lam, J. Y., Wong, W. M., Mak, L. F., Wang, X., Chu, H., Cai, J. P., Jin, D. Y., To, KK., Chan, J. F., et al. (2020). SARS-CoV-2 nsp13, nsp14, nsp15 and orf6 function as potent interferon antagonists. Emerg Microbes Infect 9, 1418-1428.

Zhou, P., Yang, X. L., Wang, X. G., Hu, B., Zhang, L., Zhang, W., Si, H. R., Zhu, Y., Li, B., Huang, C. L., et al. (2020). A pneumonia outbreak associated with a new coronavirus of probable bat origin. Nature 579, 270-273.

1. A T cell receptor fusion construct comprising
(a) a peptidic moiety binding to a spike protein, wherein the spike protein is characterized in that (i) it is from a coronavirus and (ii) it binds to ACE2; and
(b) a peptidic moiety selected from the group consisting of T cell receptor α chain, T cell receptor β chain, CD3γ, CD3ε, CD3δ, and variants of any of the foregoing.

2. The T cell receptor fusion construct according to claim 1, wherein the construct further comprises a peptidic linker between the peptidic moiety (a) and the peptidic moiety (b).

3. The T cell receptor fusion construct according to claim 1 or 2, wherein the construct further comprises a signal peptide before the peptidic moiety (a).

4. The T cell receptor fusion construct according to any one of claims 1 to 3, wherein the peptidic moiety (b) comprises an extracellular region, a transmembrane region and an intracellular region.

5. The T cell receptor fusion construct according to any one of claims 1 to 8, wherein the peptidic moiety (a) is selected from the group consisting of an antibody-derived fragment, an aptamer, and a receptor or a binding domain thereof.

6. The T cell receptor fusion construct according to claim 5, wherein the peptidic moiety (a) is a receptor or a binding domain thereof and wherein the receptor is ACE2.

7. The T cell receptor fusion construct according to any one of claims 1 to 6, wherein the peptidic moiety (a) comprises the amino acid sequence of SEQ ID NO: 2 or a sequence that is at least 85% identical thereto.

8. The T cell receptor fusion construct according to claim 5, wherein the peptidic moiety (a) is an antibody-derived fragment and wherein the antibody-derived fragment is a single chain fragment (scFv).

9. The T cell receptor fusion construct according to claim 8, wherein the single chain fragment comprises the amino acid sequence of SEQ ID NO: 5 and the amino acid sequence of SEQ ID NO: 6.

10. The T cell receptor fusion construct according to claim 9, wherein the single chain fragment (scFv) further comprises a peptidic linker between the amino acid sequences of SEQ ID NO: 5 and SEQ ID NO: 6.

11. The T cell receptor fusion construct according to any one of claims 1 to 10, wherein the spike protein is characterized in that it is from a coronavirus selected from the group consisting of SARS-CoV-2, SARS-CoV-1 and HCoV-NL63.

12. The T cell receptor fusion construct according to any one of claims 1 to 10 wherein the spike protein is characterized in that it is from SARS-CoV-2.

13. The T cell receptor fusion construct according to any one of claims 1 to 10, wherein the spike protein is characterized in that it is from SARS-CoV-1.

14. The T cell receptor fusion construct according to any one of claims 1 to 10, wherein the spike protein is characterized in that it is from HCoV-NL63.

15. A vector comprising the genetic information encoding a T cell receptor fusion construct according to any one of claims 1 to 14.

16. The vector according to claim 15, wherein the vector is selected from the group consisting of a lentiviral vector, a DNA vector, an RNA vector, a plasmid vector, a cosmid vector, a herpes virus vector, a measles virus vector, an adenoviral vector, and a retrovirus.

17. A process of transfecting or transducing T cells with a vector according to claim 15 or 16, wherein the T cells are collected and cultivated ex vivo, thereafter transfected or transduced with a vector according to claim 15 or 16, and thereafter grown and expanded ex vivo.

18. A modified T cell comprising a T cell receptor fusion construct according to any one of claims 1 to 14.

19. The T cell receptor fusion construct according to any one of claims 1 to 14, or the vector according to claim 15 or 16, or the modified T cell according to claim 18 for use in the treatment of a disease, preferably for use in the treatment of a disease caused by a coronavirus.

20. The T cell receptor fusion construct according to claim 12, or a vector comprising the genetic information encoding a T cell receptor fusion construct according to claim 12, or a modified T cell comprising a T cell receptor fusion construct according to claim 12 for use in the treatment of the coronavirus 2019 (COVID-19) disease.

21. The T cell receptor fusion construct according to claim 13, or a vector comprising the genetic information encoding a T cell receptor fusion construct according to claim 13, or a modified T cell comprising a T cell receptor fusion construct according to claim 13 for use in the treatment of the severe acute respiratory syndrome (SARS) disease.

22. The T cell receptor fusion construct according to claim 14, or a vector comprising the genetic information encoding a T cell receptor fusion construct according to claim 14, or a modified T cell comprising a T cell receptor fusion construct according to claim 14 for use in the treatment of symptoms associated with a HCoV-NL63 infection.
